SEQUENCE LISTING (8 sequences)

SEQ ID NO: 1 - DNA, 333 bp, Eggplant latent viroid
gggtggtgtgtgccacccctgatgagaccgaaaggtcgaaatggggtttcgccatgggtcgggactttaaattcggaggattcgtcctttaaacgttcctccaagagtcccttccccaaacccttactttgtaagtgtggttcggcgaatgtaccgtttcgtcctttcggactcatcagggaaagtacacactttccgacggtgggttcgtcgacacctctccccctcccaggtactatcccctttcaaggatgtgttccctaggagggtgggtgtacctcttttggattgctccggccttccaggagagatagaggacgacctctccccata

SEQ ID NO: 2 - DNA, 247 bp, Avocado sunblotch viroid
tttattagaacaagaagtgaggatatgattaaactttgtttgacgaaaccaggtctgttccgactttccgactctgagtttcgacttgtgagagaaggaggagtcgtggtgaacttttattaaaaaaattagttcactcgtcttcaatctcttgatcacttcgtctcttcagggaaagatgggaagaacactgatgagtctcgcaaggtttactcctctatcttcattgtttttttacaaaatcttg

SEQ ID NO: 3 - DNA, 399 bp, Chrysanthemum chlorotic mottle viroid
ggcacctgacgtcggtgtcctgatgaagatccatgacaggatcgaaacctcttccagtttcggcttgtgtgggagtaaagctttcgctctctccacagcctcatcaggaaacccacttcaggtctcgactggaaggtcgttaaacttcccctctaagcggagtagaggtaaatacctccgtccaaccccgggaggaaaggggttgggacccggaacagatccagttccggtcctttggagtccatttctctcgttggatattctcctcggagaagggagatggggccagtcccagtcggttcgctctcgtagtcacagccactggggaacctaggcagatggctggacggagtcttagtccactccagaggaccttgggtttgaaacccccaagaggtc

SEQ ID NO: 4 - DNA, 337 bp, Peach latent mosaic viroid
gtcataagtttcgtcgcatttcagcgactcatcagtgggcttagcccagacttatgagagagtaaagacctctcagcccctccaccttggggtgccctattcggagcactgcagttcccgatagaaaggctaagcacctcgcaatgaggtaaggtgggacttttccttctggaaccaagcggttggttccgaggggggtgtgatccaggtaccgccgtagaaactggattacgacgtctacccgggattcaaacccgtcccctccagaagtgattctggatgaagagtctgtgctaagcacactgacgagtctctgagatgagacgaaactcttctt

SEQ ID NO: 5 - protein, 1167 aa, Solanum melongena
MSVSHRVIYSFTHYKLYNLSSSLSSLPSRIFFPFQSPSFHTFSSLMPNNQERGGYEGKKWQVRPSSNRVPGSSSNVEPVSAATAEAITDRLKSVDITESGAQSSVPVTSLQFGSVGLAPQSPVQHQKVIWKPKSYGTVSGAPVVEAGKTPVEQKSALLSKLFKGNLLENFTVDNSTFSRAQVRATFYPKFENEKSDQEIRTRMIEMVSKGLAIVEVTLKHSGSLFMYAGHEGGAYAKNSFGNIYTAVGVFVLGRMFREAWGTKASKKQAEFNEFLERNRMCISMELVTAVLGDHGQRPRDDYAVVTAVTELGNGKPTFYSTPDVIAFCREWRLPTNHVWLFSTRKSVTSFFAAYDALCEEGTATTVCEALSEVADISVPGSKDHIKVQGEILEGLVARIVKRESSEHMERVLRDFPPPPSEGEGLDLGPTLREICAANRSEKQQIKALLQSAGTAFCPNYLDWFGDENSGSHSRNADRSVVSKFLQSHPADLYTGKIQEMVRLMREKRFPAAFKCHYNLHKINDVSSNNLPFKMVIHVYSDSGFRRYQKEMRHKPGLWPLYRGFFVDLDLFKVNEKKTAEMAGSNNQMVKNVEEDNSLADEDANLMVKMKFLTYKLRTFLIRNGLSTLFKEGPSAYKSYYLRQMKIWNTSAAKQRELSKMLDEWAVYIRRKYGNKPLSSSTYLSEAEPFLEQYAKRSPQNHALIGSAGNFVKVEDFMAIVEGEDEEGDLEPAKDIAPSSPSISTRDMVAKNEGLIIFFPGIPGCAKSALCKEILNAPGGLGDDRPVNSLMGDLIKGRYWQKVADERRRKPYSIMLADKNAPNEEVWKQIENMCLSTGASAIPVIPDSEGTETNPFSIDALAVFIFRVLHRVNHPGNLDKSSPNAGYVMLMFYHLYDGKSRQEFESELIERFGSLVRIPVLKPERSPLPDSVRSIIEEGLSLYRLHTTKHGRLESTKGTYVQEWVKWEKQLRDILLGNADYLNSIQVPFEFAVKEVLEQLKVIARGEYAVPAEKRKLGSIVFAAISLPVPEILGLLNDLAKKDPKVGDFIKDKSMESSIQKAHLTLAHKRSHGVTAVANYGSFLHQKVPVDVAALLFSDKLAALEAEPGSVEGEKINSKNSWPHITLWSGAGVAAKDANTLPQLLSQGKATRIDINPPVTITGTLEFF

SEQ ID NO: 6 - DNA, 2050 bp, Artificial sequence
  Synthetic sequence Sequence of plasmid pLELVd
cgatgcttctttgagcgaacgatcaaaaataagtgccttcccatcaaaaaaatattctcaacataaaaaactttgtgtaatacttgtaacgctgccccatagggtggtgtgtgccacccctgatgagaccgaaaggtcgaaatggggtttcgccatgggtcgggactttaaattcggaggattcgtcctttaaacgttcctccaagagtcccttccccaaacccttactttgtaagtgtggttcggcgaatgtaccgtttcgtcctttcggactcatcagggaaagtacacactttccgacggtgggttcgtcgacacctctccccctcccaggtactatcccctttcaaggatgtgttccctaggagggtgggtgtacctcttttggattgctccggccttccaggagagatagaggacgacctctccccatagggtggtgtgtgccacccctgatgagaccgaaaggtcgaaatgggggaaatcatccttagcgaaagctaaggattttttttatctgaaatgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcat

SEQ ID NO: 7 - DNA, 2148 bp, Artificial sequence
  Synthetic sequence Sequence of plasmid pLELVd-Spinach
cgatgcttctttgagcgaacgatcaaaaataagtgccttcccatcaaaaaaatattctcaacataaaaaactttgtgtaatacttgtaacgctgccccatagggtggtgtgtgccacccctgatgagaccgaaaggtcgaaatggggtttcgccatgggtcgggactttaaattcggaggattcgtcctttaaacgttcctccaagagtcccttccccaaacccttactttgtaagtgtggttcggcgaatgtaccgtttcgtcctttcggactcatcagggaaagtacacactttccgacggtgggttcgtcgacacctctccccctcccaggtactatccccttgacgcaactgaatgaaatggtgaaggacgggtccaggtgtggctgcttcggcagtgcagcttgttgagtagagtgtgagctccgtaactagtcgcgtctcaaggatgtgttccctaggagggtgggtgtacctcttttggattgctccggccttccaggagagatagaggacgacctctccccatagggtggtgtgtgccacccctgatgagaccgaaaggtcgaaatgggggaaatcatccttagcgaaagctaaggattttttttatctgaaatgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcat

SEQ ID NO: 8 - DNA, 2092 bp, Artificial sequence
  Synthetic sequence Sequence of plasmid pLELVd-Strep
cgatgcttctttgagcgaacgatcaaaaataagtgccttcccatcaaaaaaatattctcaacataaaaaactttgtgtaatacttgtaacgctgccccatagggtggtgtgtgccacccctgatgagaccgaaaggtcgaaatggggtttcgccatgggtcgggactttaaattcggaggattcgtcctttaaacgttcctccaagagtcccttccccaaacccttactttgtaagtgtggttcggcgaatgtaccgtttcgtcctttcggactcatcagggaaagtacacactttccgacggtgggttcgtcgacacctctccccctcccaggtactatccccttccgaccagaatcatgcaagtgcgtaagatagtcgcgggccggtcaaggatgtgttccctaggagggtgggtgtacctcttttggattgctccggccttccaggagagatagaggacgacctctccccatagggtggtgtgtgccacccctgatgagaccgaaaggtcgaaatgggggaaatcatccttagcgaaagctaaggattttttttatctgaaatgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcat

(19) United States
(12) Patent Application Publication
(10) Pub. No.: US 2017/0088871 A1
(43) Pub. Date: Mar. 30, 2017

(54) RECOMBINANT RNA PRODUCTION

(71) Applicants: Consejo Superior de Investigaciones Científicas, Madrid (ES); Universitat Politècnica de València, València (ES)

(72) Inventors: José-Antonio DARÒS, Valencia (ES); Verónica ARAGONÉS, Valencia (ES); María-Teresa CORDERO, Valencia (ES)

(21) Appl. No.: 15/312,552

(22) PCT Filed: May 18, 2015
(86) PCT No.: PCT/EP2015/060912

§ 371 Date: Nov. 18, 2016

(30) Foreign Application Priority Data
May 20, 2014 (EP) 14382177.5

(51) Int. Cl. C12P 19/34; C12N 7/00; C12N 9/00; C12N 15/70

(52) U.S. Cl. CPC C12P 19/34; C12N 15/70; C12N 7/00; C12N 9/93; C12Y 601/01; C12N 2790/14043

(57) ABSTRACT

This invention relates to the production of RNA by co-expressing a tRNA ligase and a chimeric RNA molecule comprising a target RNA and a plant viroid scaffold, such as Eggplant latent viroid, in a host cell. This co-expression causes the production of large amounts of the chimeric RNA molecule in the host cells and may be useful for example in the production of RNA aptamers and other RNA molecules.

### FIELD

This invention relates to the production of recombinant RNA.

### BACKGROUND

Despite initial relegation to a secondary role, current knowledge illustrates how RNA molecules play crucial functions in most biological processes (Holt and Schuman, 2013; Sabin et al., 2013), perhaps as a result of their remarkable conformation plasticity and selectivity (Nakamura et al., 2012). Properties that, in combination with a straight-forward mechanism of replication based on base complementarity, most possibly determined the decisive role of RNA in evolution of life (Gold et al., 2012). The versatility of RNA has also made possible engineering artificial molecules to carry on novel functions in different approaches of synthetic biology (Breaker, 2004; Isaacs et al., 2006; Rodrigo et al., 2013). This is the case, for example, of RNA aptamers, RNA oligonucleotides able to bind targets with high affinity and specificity, which are typically selected through a process known as systematic evolution of ligands by exponential enrichment (SELEX) (Ellington and Szostak, 1990; Robertson and Joyce, 1990; Tuerk and Gold, 1990), and have enormous potential applications in biotechnology (Zhou et al., 2012; Germer et al., 2013). However, in contrast to what occurs with other central players of biological processes, like DNA or proteins, the intrinsic low half-life of RNA limits the possibility of efficient overproduction of recombinant RNA molecules (Ponchon and Dardel, 2011), restricting the advance of RNA biotechnology.

RNA molecules play crucial roles in most biological processes and are currently envisioned as feasible targets for therapeutic approaches (Roberts and Wood, 2013). Symmetrically, engineered RNA molecules have an enormous potential as therapeutic agents due to their ability to bind proteins (and other cellular components) and specifically regulate their functions with minimal or no harmful side-effects (Sundaram et al., 2013). In addition, RNA aptamers can be easily selected to bind many types of targets, including proteins, DNA, RNA, metabolites, small organic compounds, viruses, and even whole cells, which opens unlimited perspectives of biotechnological applications of this type of molecules (Citartan et al., 2012; Gold et al., 2012). However, research and biotechnological applications of RNA are currently restricted in part by the difficulties to produce the large amounts of recombinant RNAs that these developments require.

Conventional strategies to produce large amounts of RNA molecules include chemical synthesis or in vitro transcription (Milligan et al., 1987). However, recent new initiatives have proposed the use of E. coli as an alternative cost-effective biofactory to overproduce large amounts of recombinant RNAs (Ponchon and Dardel, 2011; Batey, 2014). Most strategies to overproduce recombinant RNAs in E. coli are based on expression of chimeric molecules consisting of the RNA of interest embedded into well-structured endogenous RNA molecules or domains that are particularly stable and accumulate to high levels by themselves, like tRNAs (Ponchon and Dardel, 2007; Ponchon et al., 2013), RNase P RNA (Paige et al., 2011), transfer-messenger RNA domains (Umekage and Kikuchi, 2009b) or rRNA variants (Umekage and Kikuchi, 2009a). These RNA molecules act as scaffolds in the RNA expression process (Ponchon et al., 2009). The scaffold part of the chimeric recombinant RNA is properly processed by the bacterial machinery and contributes to the intracellular stability of the whole product.

### SUMMARY

The present invention results from the unexpected finding that large amounts of a target RNA are produced in host cells that express a modified plant viroid in which the coding sequence of the target RNA is inserted. This may be useful for example in the production of aptamers, miRNAs and other RNA molecules.

An aspect of the invention provides a method of RNA production comprising;
expressing in a host cell;
a nucleic acid encoding a chimeric RNA molecule comprising a target RNA and a plant viroid scaffold; and,
a nucleic acid encoding a tRNA ligase,
such that the chimeric RNA molecule is produced in the host cells.

Other aspects of the invention provide nucleic acids, vectors, host cells, systems and kits suitable for use in the methods of RNA production.

### BRIEF DESCRIPTION OF FIGURES

FIG. 1 shows the sequence and predicted structure of minimum free energy of Eggplant latent viroid (ELVd; GenBank accession number AJ536613). The hammerhead ribozyme domain and self-cleavage site are indicated on yellow background and by a red arrowhead, respectively. The position U245-U246 where recombinant RNAs were inserted (see below) is indicated by an arrow.

FIG. 2 shows analysis of RNA accumulating in E. coli co-expressing ELVd RNA and eggplant tRNA ligase. RNA was separated by single denaturing PAGE (A and B) or two dimensional PAGE (C and D), first dimension in the presence of 89 mM TBE and second in the presence of 22.5 mM TBE, as indicated by arrows. Gels were stained with ethidium bromide, the RNA in the gels transferred to membranes, and the membranes hybridized with a radioactive probe complementary to ELVd RNA. (A and C) Gels stained with ethidium bromide. (B and D) Autoradiographies of the hybridized membranes. (A and B) Lane 1, RNA marker with the sizes of the standards indicated on the left in nt; lane 2, RNA from E. coli transformed with pLELVd; lane 3, RNA from E. coli co-transformed with pLELVd and p15tRnlSm; lane 4, RNA from E. coli transformed with p15tRnlSm. The positions of two E. coli ribosomal RNAs and the circular and linear ELVd RNAs are indicated on the right of both panels. (C and D) RNA from E. coli co-transformed with pLELVd and p15tRnlSm. The positions of the circular and linear ELVd RNAs are indicated inside the panels. E. coli cultures were grown at 25° C. and induced with 0.4 mM IPTG at DO₆₀₀=0.6. Bacteria were harvested at 10 h post-induction. Each lane contains an RNA aliquot corresponding to 0.8 ml of culture.

FIG. 3 shows analysis of RNA accumulating in E. coli co-expressing ELVd RNA and eggplant tRNA ligase or mCherry. RNA from three independent clones of each type was separated by denaturing PAGE and the gel stained with ethidium bromide. Lane 1, RNA marker with the sizes of the standards indicated on the left in nt; lanes 2 to 4, RNA from three E. coli clones transformed with pLELVd; lanes 5 to 7, RNA from three E. coli clones co-transformed with pLELVd and p15tRnlSm; lanes 8 to 10, RNA from three E. coli clones co-transformed with pLELVd and p15tRnlSm. The positions of the circular and linear ELVd RNAs are indicated on the right. E. coli cultures were grown at 37° C. and induced with 0.1 mM IPTG at DO₆₀₀=0.1. Bacteria were harvested at 8 h post-induction. Each lane contains an aliquot of RNA corresponding to 0.8 ml of culture.

FIG. 4 shows a time-course analysis of RNA accumulating in E. coli co-expressing ELVd RNA and eggplant tRNA ligase, grown at different temperatures. (A and B) Cultures grown at 25 and 37° C., respectively. Aliquots of the cultures were taken at the indicated time points after induction, the RNA purified and analyzed by denaturing PAGE and ethidium bromide staining of the gel. Lanes 1, RNA marker with the sizes of the standards indicated on the left in nt; lanes 2 to 15, RNA from aliquots of the cultures taken at 0, 1, 2, 3, 4, 5, 6, 7, 8, 9, 10, 11, 12 and 22 h post-induction. The positions of the circular and linear ELVd RNAs are indicated on the right. E. coli cultures were induced with 0.4 mM IPTG at DO₆₀₀=0.6. Each lane contains an aliquot of RNA corresponding to 0.8 ml of culture.

FIG. 5 shows an analysis of RNA accumulating in E. coli co-expressing ELVd RNA and eggplant tRNA ligase, induced with different amounts of IPTG. RNA preparations were separated by denaturing PAGE and the gel stained with ethidium bromide. Lanes 1, RNA marker with the sizes of the standards indicated on the left in nt; lanes 2, RNA from a non-induced culture; lanes 3 to 7, RNAs from cultures induced with 0.1, 0.2, 0.4, 0.8 and 1.6 mM IPTG, respectively. The positions of the circular and linear ELVd RNAs are indicated on the right. E. coli cultures were grown at 37° C. and induced at DO₆₀₀=0.6. Bacteria were harvested at 6 h post-induction. Each lane contains an aliquot of RNA corresponding to 0.8 ml of culture.

FIG. 6 shows a time-course analysis of RNA accumulating in E. coli co-expressing ELVd RNA and eggplant tRNA ligase, induced at different OD₆₀₀. Aliquots were taken from the cultures at different time points, and RNA extracted and analyzed by denaturing PAGE and ethidium bromide staining. Lanes 1 to 7, RNAs from the culture induced at OD₆₀₀=0.6 taken at 0, 2, 3, 4, 5, 6 and 7 h post-induction; lane 8, RNA marker with the sizes of the standards indicated on the left in nt; lanes 9 to 16, RNAs from the culture induced at OD600=0.1 taken at 0, 2, 4, 5, 6, 7, 8 and 9 h post-induction. The positions of the circular and linear ELVd RNAs are indicated on the right. E. coli cultures were grown at 37° C. and induced with 0.1 mM ITPG. Each lane contains an aliquot of RNA corresponding to 0.4 ml of culture.

FIG. 7 shows time-course analysis of RNA accumulating in E. coli co-expressing ELVd RNA and eggplant tRNA ligase, grown in LB and TB media. Aliquots were taken from the cultures at different time points, RNA extracted and analyzed by denaturing PAGE and ethidium bromide staining. Lanes 1 to 8, RNAs from the culture grown in LB medium taken at 0, 2, 4, 6, 8, 10, 12 and 24 h post-induction; lanes 9 to 16, RNAs from the culture grown in TB medium taken at 0, 2, 4, 6, 8, 10, 12 and 24 h post-induction. The positions of the circular and linear ELVd RNAs are indicated on the right. E. coli cultures were grown at 37° C. and induced with 0.1 mM ITPG at DO₆₀₀=0.1 mM. Each lane contains an aliquot of RNA corresponding to 0.4 ml of culture.

FIG. 8 shows a time-course analysis of RNA accumulating in E. coli co-expressing ELVd RNA and eggplant tRNA ligase in optimal conditions. Aliquots from the culture were taken at different time points and RNA purified. RNAs were separated by denaturing PAGE and the gel stained with ethidium bromide. Lanes 1 to 8, RNAs from aliquots taken at 0, 2, 4, 6, 8, 10, 12 and 24 h post-induction. The positions of the circular and linear ELVd RNAs are indicated on the right. E. coli cultures were grown at 37° C. in TB medium and induced with 0.1 mM ITPG at DO₆₀₀=0.1 mM. Each lane contains an aliquot of RNA corresponding to 0.4 ml of culture.

FIG. 9 shows quantification of the ELVd RNA produced with the E. coli system co-expressing ELVd RNA and eggplant tRNA ligase. The RNA extracted from an aliquot of the culture taken at 12 h post-induction was subjected to serial dilutions. Samples were separated by denaturing PAGE and the gel stained with ethidium bromide. Lane 1, RNA marker with the sizes of the standards indicated on the left in nt; lanes 2 to 8, RNA serial dilutions corresponding to 400, 80, 16, 3.2, 0.64, 0.13 and 0.03 μl of the original culture, respectively. The positions of the circular and linear ELVd RNAs are indicated on the right. E. coli cultures were grown at 37° C. in TB medium and induced with 0.1 mM ITPG at DO₆₀₀=0.1 mM.

FIG. 10 shows pilot purification or recombinant ELVd from an E. coli culture. Total RNA from bacterial cells was purified by phenol:chloroform extraction and chromatographed through a DEAE Sepharose column (chromatogram is shown). RNA in elution fractions were analyzed by denaturing PAGE and ethidium bromide staining (gel is shown). RNA in peak eluting fraction was electrophoresed in a 5% polyacrylamide, 8 M urea, 1×TBE gel. After ethidium bromide staining (bottom gel on the left), the band corresponding to the circular ELVd RNA was cut and loaded onto a second gel that was run in native conditions (5% polyacrylamide, TAE). Ethidium bromide staining of the second gel (bottom gel on the right) shows monomeric circular ELVd RNA purified to homogeneity.

FIG. 11 shows time-course analysis of RNA accumulating in E. coli co-expressing ELVd-Spinach RNA and eggplant tRNA ligase. Aliquots from the culture were taken at different time points and RNA purified. RNAs were separated by denaturing PAGE and the gel stained with ethidium bromide. Lanes 1 to 8, RNAs from aliquots taken at 0, 2, 4, 6, 8, 10, 12 and 24 h post-induction. The positions of the circular and linear ELVd-Spinach RNAs are indicated on the right. E. coli cultures were grown at 37° C. in TB medium and induced with 0.1 mM ITPG at DO₆₀₀=0.1 mM. Each lane contains an aliquot of RNA corresponding to 0.4 ml of culture.

FIG. 12 shows quantification of the ELVd-Spinach hybrid RNA produced with the E. coli to overexpress recombinant RNA. The RNA extracted from an aliquot of the culture taken at 14 h post-induction was subjected to serial dilutions. Samples were separated by denaturing PAGE and the gel stained with ethidium bromide. Lanes 1 to 6, RNA serial dilutions corresponding to 400, 80, 16, 3.2, 0.64 and 0.13 μl of the original culture, respectively. The positions of the circular and linear ELVd-Spinach RNAs are indicated on the right. E. coli cultures were grown at 37° C. in TB medium and induced with 0.1 mM ITPG at DO₆₀₀=0.1 mM.

FIG. 13 shows fluorescence emission of ELVd-Spinach hybrid RNA. Photographs taken under UV illumination using a GFP filter. (A) Aliquots of independent E. coli cultures co-expressing tRNA ligase and ELVd (upper) or ELVd-Spinach (lower) RNAs were incubated with DFHBI and the cells sedimented. (B) DFHBI was added to RNA extracts from E. coli cultures co-expressing tRNA ligase and ELVd (left) or ELVd-Spinach (right).

FIG. 14 shows analysis of RNA accumulating in E. coli co-expressing ELVd-Strep RNA and eggplant tRNA ligase. RNA preparations were separated by denaturing PAGE and the gel stained with ethidium bromide. Lanes 1, RNA marker with the sizes of the standards indicated on the left in nt; lanes 2 to 4, E. coli clones expressing ELVd RNA; lanes 5 to 7, E. coli clones expressing ELVd-Strep. The positions of the corresponding circular and linear RNA forms are indicated by red and blue arrowheads, respectively. E. coli cultures were grown at 37° C. in TB medium and induced at DO₆₀₀=0.1 with 0.1 mM IPTG. Bacteria were harvested at 14 h post-induction. Each lane contains an aliquot of RNA corresponding to 0.4 ml of culture.

FIG. 15 shows a scheme of the deletions created on the ELVd RNA molecule and assayed in the E. coli system to overproduce recombinant RNA. (A) Deleted sequences I1, I2, I3, I4, D1, D2, D3 and D4 are indicated on the ELVd conformation of minimum free energy by lines of different colours. (B) Schemes of the conformations of minimum free energy of ELVd and the different ELVd deleted forms.

FIG. 16 shows analysis of RNA accumulating in E. coli co-expressing deleted forms of ELVd RNA and eggplant tRNA ligase. Aliquots of the cultures were taken at 8 h post-induction, RNA purified from the cells and separated by (A and B) single denaturing PAGE or (C) two dimensional denaturing PAGE, first in TBE and second in 0.25×TBE. The gels were stained with ethidium bromide. (A) Lane 1, wild-type ELVd; lanes 2 and 3, ELVd deleted form I1; lanes 4 and 5, ELVd deleted form I2; lanes 6 and 7, ELVd deleted form I3; lanes 8 and 9, ELVd deleted form I4. (B) Lane 1, wild-type ELVd; lanes 2 and 3, ELVd deleted form D1; lanes 4 and 5, ELVd deleted form D2; lanes 6 and 7, ELVd deleted form D3; lanes 8 and 9, ELVd deleted form D4. The positions of the circular and linear ELVd RNAs are indicated on the left of both panels. The positions of the corresponding circular and linear RNAs are indicated by red and blue arrowheads, respectively. (C) Wild-type ELVd and ELVd deleted forms I1, I2, I3, I4, D1, D2, D3 and D4, as indicated. The directions of migration of the two electrophoretic separations are indicated. The positions of the corresponding circular RNAs are indicated by a red arrowhead. E. coli cultures were grown at 37° C. in LB medium and induced at DO₆₀₀=0.1 with 0.1 mM IPTG. Bacteria were harvested at 8 h post-induction. Each lane contains an aliquot of RNA corresponding to 0.8 ml of culture.

FIG. 17 shows analysis of RNA accumulating in E. coli co-expressing double-deleted forms of ELVd RNA and eggplant tRNA ligase. Aliquots of the cultures were taken at 8 h post-induction, RNA purified from the cells and separated by (A) single denaturing PAGE or (B) two dimensional denaturing PAGE, first in TBE and second in 0.25×TBE. The gels were stained with ethidium bromide. (A) Lane 1, RNA marker with the sizes of the standards indicated on the left in nt; lanes 2 and 3, wild-type ELVd; lanes 4 and 5, ELVd double-deleted form I1D3; lanes 6 and 7, ELVd double-deleted form I2D2; lanes 8 and 9, ELVd double-deleted form I3D1; lanes 10 and 11, ELVd double-deleted form I3D3. The positions of the corresponding circular and linear RNAs are indicated by red and blue arrowheads, respectively. (B) Wild-type ELVd and ELVd double-deleted forms I1D3, I2D2, I3D1 and I3D3, as indicated. The directions of migration of the two electrophoretic separations are indicated. The positions of the corresponding circular RNAs are indicated by a red arrowhead. E. coli cultures were grown at 37° C. in LB medium and induced at DO₆₀₀=0.1 with 0.1 mM IPTG. Bacteria were harvested at 8 h post-induction. Each lane contains an aliquot of RNA corresponding to 0.8 ml of culture.

FIG. 18 shows quantification of the ELVd I3D1-Spinach hybrid RNA produced with the E. coli system to overexpress recombinant RNA. The RNA extracted from an aliquot of the culture taken at 14 h post-induction was subjected to serial dilutions. Samples were separated by denaturing PAGE and the gel stained with ethidium bromide. Lanes 1 to 6, RNA serial dilutions corresponding to 400, 80, 16, 3.2, 0.64 and 0.13 μl of the original culture, respectively. The positions of the circular and linear ELVd I3D1-Spinach RNAs are indicated on the right. E. coli cultures were grown at 37° C. in TB medium and induced with 0.1 mM ITPG at DO₆₀₀=0.1 mM.

FIG. 19 shows time-course analysis of RNA accumulating in E. coli co-expressing ELVd RNA and eggplant tRNA ligase under the control of (A) T7 bacteriophage RNA polymerase and (B) E. coli murein lipoprotein promoters. Aliquots from the cultures were taken at different time points and RNAs purified. RNAs were separated by denaturing PAGE and the gels stained with ethidium bromide. Lanes 1, RNA marker with the sizes of the standards indicated on the left in nt; lanes 2 to 11, RNAs from aliquots taken at 0, 1, 2, 3, 4, 5, 6, 7, 8 and 9 h post-induction of the first culture. The positions of the circular and linear ELVd-Spinach RNAs are indicated on the right. E. coli cultures were grown at 37° C. in TB medium and induced with 0.1 mM IPTG (A) or not (B) at DO₆₀₀=0.1 mM. Each lane contains an aliquot of RNA corresponding to 0.2 ml of culture.

FIG. 20 shows the production of two chimeric RNAs with potential anti-HCV activity in E. coli using the ELVd-derived system. RNAs were purified from E. coli cultures, separated by denaturing PAGE and the gel stained with ethidium bromide. Lane 1, RNA marker with the sizes of the standards indicated on the left in nt; lanes 2 to 4, serial dilutions (1/5) of the sample corresponding to the ELVdI3D1 control (empty viroid); lanes 5 to 7 and 8 to 10, serial dilutions (1/5) of the samples corresponding to ELVdI3D1-HCV1 (lanes 5 to 7) and ELVdI3D1-HCV1 (lanes 8 to 10). Arrows point to the circular forms of the RNAs of interest. E. coli cultures were grown at 37° C. in TB medium during 8 h. Lanes 2, 5 and 8 were loaded with the RNAs purified from the equivalent to 0.4 ml of culture. Lanes 3, 4; 6, 7; and 9, 10 contain serial dilutions (1/5) of samples in lanes 1, 5 and 8, respectively.

FIG. 21 shows an analysis of the chimeric ELVdI3D1-miR RNAs produced in the E. coli system derived from ELVd. RNAs extracted from independent E. coli clones were separated by denaturing PAGE and the gel stained with ethidium bromide. Lane 1, RNA marker with the sizes of the standards indicated on the left in nt; lanes 2 to 4, control expressing full-length ELVd at 6 (lane 2), 7 (lane 3) and 8 h (lane 4), respectively; lanes 5 to 10 and 11 to 16, two independent clones each expressing ELVdI3D1-miR with three (lanes 5 to 10) or four (lanes 11 to 16) tandem repetitions of the miRNA target at 6 (lanes 5, 8, 11, 14), 7 (lanes 6, 9, 12, 15) and 8 h (lanes 7, 10, 13, 16), respectively. Arrows point to the circular forms of the expressed RNAs in each case. E. coli cultures were grown at 37° C. in TB medium. Each lane contains the RNAs purified from the equivalent to 0.2 ml of culture.

FIG. 22 shows selection of plasmids to produce recombinant RNAs with potential anti-insect activity in E. coli by means of the ELVd-derived system. Selected plasmids were digested with BsaI to liberate the insert and the products of the reaction separated by non-denaturing PAGE. The gel stained with ethidium bromide. Lane 1, DNA marker consisting of a 100-bp ladder; lanes 2 and 10, DNA marker consisting of a 50-bp ladder; lanes 3 to 5, plasmids to express ELVd-H1; lane 6, control plasmid to express ELVd with no insert; lanes 7 to 9, plasmids to express ELVd-H2. Arrows point to delayed bands that demonstrate cloning of the H1 and H2 cDNAs.

FIG. 23 shows the purification by size exclusion chromatography of recombinant RNA produced in E. coli using the viroid-derived system. Aliquots of the RNA fractionated with a Superdex 200 10/300 GL column were separated by denaturing PAGE and the gel stained with ethidium bromide. Lane 1, RNA marker with the sizes of the standards indicated on the left in nt; lane 2, aliquot of the total RNA loaded to the column; lanes 3 to 16, aliquots of fractions 6 to 19 as eluted from the column. Arrow points to a fraction in which the recombinant RNA is reasonably separated from contaminating E. coli RNAs. Analyzed aliquots correspond to 10 μl of the 1-ml fractions obtained in the chromatography.

### DETAILED DESCRIPTION

The invention relates to the production of recombinant RNA in host cells through the co-expression of a tRNA ligase and a chimeric RNA molecule that comprises a target RNA within a plant viroid scaffold. Co-expression of the tRNA ligase and the chimeric RNA molecule produces large amounts of monomeric chimeric RNA molecule in the host cells. The amount of monomeric chimeric RNA produced is significantly greater (e.g. orders of magnitude greater) than the amounts of RNA produced by prior art RNA expression systems, for example using tRNA or rRNA scaffolds.

In some embodiments, more than 10 mg, more than 20 mg or more than 30 mg of monomeric chimeric RNA may be produced per litre of the host cell culture. For example, the data herein shows the production of 150 mg ELVd RNA, 75 mg ELVd-Spinach RNA and 30 mg ELVdI3D1-Spinach RNA per litre of E. coli culture.

Without being bound by theory, the high levels of RNA production observed using the methods described herein may result from the selection by evolution of plant viroids to survive in a hostile intracellular environment following infection. Although the circular viroid scaffold may contribute to RNase resistance, the data herein also shows that co-expression with tRNA ligase is important in the huge accumulation of RNA. This may arise from preservation of the chimeric RNA molecules in RNA-protein complexes (i.e. ribonucleoprotein complexes that comprise the chimeric RNA molecule and the tRNA ligase).

A range of host cells suitable for the production of RNA as described herein are known in the art. Suitable host cells may include prokaryotic cells, in particular bacteria such as P. fluorescens and Escherichia coli, and plant or non-plant eukaryotic cells, including yeasts, such as S. cerevisiae and P. pastoris, insect cells and mammalian cells, such as Chinese Hamster Ovary (CHO) cells and Chinese Hamster Ovary (HEK) cell.

The chimeric RNA molecule comprises a target RNA and a plant viroid scaffold.

The exogenous target RNA may be located at any position within the plant viroid scaffold that does not disrupt viroid activity. For example, the target RNA sequence may be located outside the viroid hammerhead ribozyme domain of the plant viroid scaffold. The viroid hammerhead ribozyme domain may be located at a position within the plant viroid that corresponds to bases 327 to 46 (plus) and bases 153-203 (minus) of the ELVd genome.

In some embodiments, the target RNA may be position within the terminal loop of the upper-right hairpin of the plant viroid, for example in the region correspond to residues 242 to 249 of ELVd.

In some preferred embodiments, the target RNA is inserted into the viroid scaffold at a position corresponding to position 245-246 of ELVd.

Other suitable insertion positions in the viroid scaffold include the position corresponding to position 129-130 of ELVd.

The chimeric RNA molecule may be non-naturally occurring.

Any RNA molecule that needs to be generated in large amounts may be used as the target RNA.

Preferably, the target RNA is not derived from a plant viroid and is not naturally associated with the plant viroid scaffold (i.e. the target RNA is heterologous).

The target RNA may be non-naturally occurring.

Suitable target RNA may include sequences up to 100 bases, up to 200 bases or up to 300 bases or more. For example, the target RNA may be 10 to 200 bases.

The target RNA may be any RNA molecule of interest. Suitable target RNAs include antisense RNA, short hairpin RNA (shRNA), interfering RNA (RNAi), siRNA, miRNA, RNA aptamers, ribozymes, viral RNA, ribosomal RNA or nucleolar RNA.

In some preferred embodiments, the target RNA is an RNA aptamer.

An RNA aptamer is a non-naturally occurring RNA molecule that binds with high affinity and specificity to a molecular target, such as a small organic molecule, toxin, peptide or protein. RNA aptamers may be typically 12 to 100 bases long. RNA aptamers specific for a range of different target molecules are well-known in the art (see for example, Li et al Curr Med Chem. 2013 20(29):3655-63; Meyer et al (2011) J. Nucl Acid Article ID 904750; Germer et al (2013) Int J Biochem Mol Biol 2013; 4(1):27-40; Zhou J, et al Front Genet. 2012 3: 234; Sundaram P et al (2013). Eur. J. Pharm. Sci. 48: 259-271; Ni et al Curr Med Chem. 2011; 18(27):4206-14; Thiel et al Oligonucleotides. 2009 September; 19(3):209-22; Dausse et al Curr Opin Pharmacol. 2009 October; 9(5):602-7; Gold et al., 2012; Aquino-Jarquin G et al. Int J Mol Sci. 2011; 12(12):9155-71). RNA aptamers have numerous applications, for example, in therapeutics and diagnostics.

Examples of suitable aptamers include Spinach; streptavidin binding aptamer (Srisawat and Engelke, 2001; Srisawat and Engelke, 2002); Pegaptanib (Macugen; Rinaldi et al Retina. 2013 February; 33(2):397-402); E10030 (Conidfi et al Int. J. Mol. Sci. 2013, 14, 6690-6719); ARC1905 (Kanwar et al Crit Rev Biochem Mol Biol. December 2011; 46(6): 459-477); EYE001 (Carrasuillo et al Invest Ophthalmol Vis Sci. 2003 January; 44(1):290-9); AS1411 (Rosenberg et al Invest New Drugs. 2014 February; 32(1):178-87); NOX-A12 (Hoellenriegel J et al Blood. 2014 Feb. 13; 123(7):1032-9); ARC1779 (Cosmi et al Curr Opin Mol Ther. 2009 June; 11(3):322); REG1/RB006 (Becker et al Curr Opin Mol Ther. 2009; 11:707-715); NU172 (Wagner-Whyte et al J Thromb Haemost (Isth Congress abstracts) 2007); and the aptamers set out in Table 1 of Sundaram et al (2013) supra and Table 1 of Germer et al (2013) supra.

The plant viroid scaffold comprises all or part of a plant viroid.

Suitable plant viroids include chloroplastic plant viroids, for example Avsunviroidae viroids, such as Avocado sunblotch viroid (ASBVd), Peach latent mosaic viroid (PLMVd), Chrysanthemum chlorotic mottle viroid (CChMVd), and Eggplant latent viroid (ELVd).

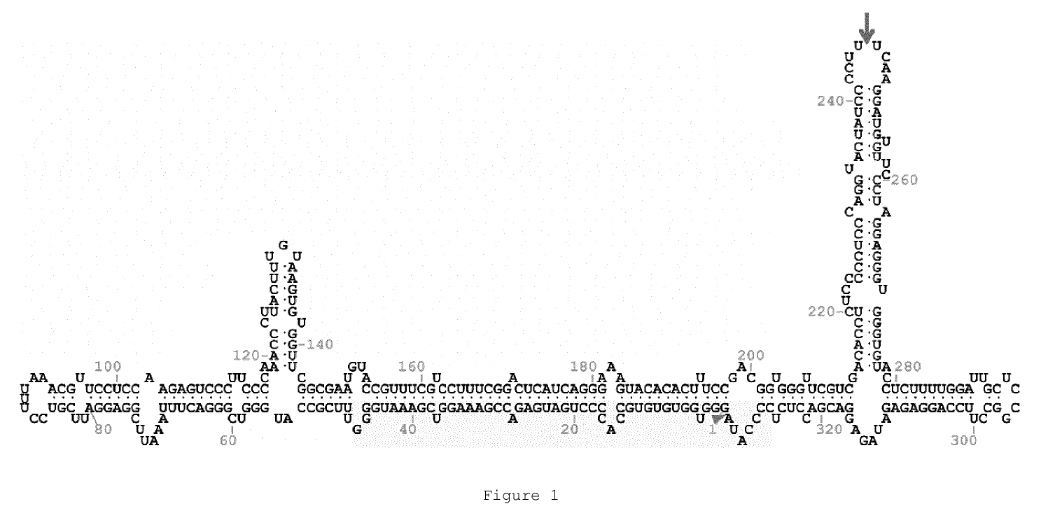

The full-length (333 base) genomic sequence of ELVd is publically available under the GenBank entry number AJ536613.1 GI: 29825431 (SEQ ID NO: 1). The sequence and structure of ELVd is shown in FIG. 1. The hammerhead ribozyme region of ELVd corresponds to bases 327 to 46 (plus) and bases 153-203 (minus).

The full-length (247 base) genomic sequence of ASBVd is publically available under the GenBank entry number NC_001410.1 GI: 11496574 (SEQ ID NO: 2).

The full-length (399 base) genomic sequence of CChMVd is publically available under the GenBank entry number NC_003540.1 GI: 20095240 (SEQ ID NO: 3).

The full-length (337 base) genomic sequence of PLMVd is publically available under the GenBank entry number NC_003636.1 GI: 20177433(SEQ ID NO: 4). The hammerhead ribozyme region of PLMVd corresponds to bases 282 to 335 (plus) and bases 2-57 (minus).

In some preferred embodiments, the plant viroid is Eggplant latent viroid (ELVd).

In some embodiments, the plant viroid scaffold may be a truncation of the full-length plant viroid sequence, for example a full-length plant viroid genome sequence of any of SEQ ID NOS: 1 to 4. For example, the plant viroid scaffold may comprise or consist of the full-length plant viroid sequence with one or more deletions. The sequence and structure of ELVd is shown in FIG. 1. Suitable plant viroid scaffolds may comprise the hammerhead ribozyme region of the plant viroid, which may for example be located from bases 327 to 46 (plus) and bases 153-203 (minus) in ELVd and at the corresponding bases in other plant viroids. Corresponding bases in other plant viroid genomes may be identified using standard sequence alignment tools.

The plant viroid scaffold may be a synthetic plant viroid scaffold that does not occur in nature.

In some embodiments, the residues corresponding to bases 56 to 116 and 214 to 310 of ELVd may be deleted in the plant viroid scaffold. For example, the scaffold may comprise bases 1 to 55, 117 to 213, and 311 to 333 of SEQ ID NO: 1 or the corresponding residues in another plant viroid sequence.

In other embodiments, the plant viroid scaffold may comprise or consist of a full-length plant viroid sequence with bases corresponding to residues with bases 56 to 141 and 279 to 310 of ELVd deleted. For example, the scaffold may comprise bases 1 to 55, 142 to 278 and 311 to 311 of SEQ ID NO: 1 or the corresponding bases in another plant viroid sequence.

Other suitable plant viroid scaffolds may be readily produced using standard techniques.

In some preferred embodiments, the plant viroid scaffold may comprise (i) the sequence of bases 1 to 55, 142 to 278 and 311 to 311 of SEQ ID NO: 1; (ii) the sequence of bases 1 to 55, 117 to 213, and 311 to 333 of SEQ ID NO: 1; (iii) the sequence of SEQ ID NO: 1; or (iv) a variant of (i), (ii) or (iii).

A variant of a reference plant viroid scaffold listed above (for example, the genomic sequence of ASBVd, PLMVd, CChMVd or ELVd (SEQ ID NOs: 1 to 4); or a truncated form thereof, as set out above) may have a nucleotide sequence having at least 75%, at least 80%, at least 85%, at least 90%, at least 95% or at least 98% sequence identity to the sequence of the reference plant viroid scaffold.

Co-expression of the chimeric RNA molecule with tRNA ligase is shown herein to lead to the high level production of chimeric RNA molecule in host cells.

The tRNA ligase may be a plant tRNA ligase, for example a plant chloroplast or plant nuclear tRNA ligase. Preferably, the tRNA ligase is a plant chloroplast tRNA ligase. (Englert et al Nucl. Acids Res. (2005) 33 (1): 388-399; Nohales et al Journal of Virology p. 8269-8276).

In other embodiments, the tRNA ligase may be from a eukaryote other than a plant. Suitable tRNA ligases may be homologues or orthologues of plant tRNA ligases and may be readily identified using standard techniques.

In some embodiments, suitable tRNA ligases may bind to the plant viroid scaffold to form a ribonucleoprotein complex.

Suitable plant tRNA ligases are well-known in the art and include Solanum melongena (eggplant) tRNA ligase. The amino acid sequence of Solanum melongena tRNA ligase is publically available under the GenBank entry number AFK76482.1 GI: 388604525 (SEQ ID NO: 2) and the nucleotide coding sequence is publically available under the GenBank entry number JX025157.1 GI: 388604524.

A tRNA ligase suitable for use in methods described herein may comprise the amino acid sequence of SEQ ID NO: 5 or a variant thereof.

For example, a suitable tRNA ligase may be an orthologue from a plant species other than Solanum melongena, for example Arabidopsis thaliana, Triticum spp or Oryza sativa. Suitable orthologues may be identified using standard sequence analysis techniques.

A variant of a reference tRNA ligase sequence, including an orthologue, may have an amino acid sequence having at least 50%, at least 60%, at least 70%, at least 75%, at least 80%, at least 85%, at least 90%, at least 95% or at least 98% sequence identity to the sequence of a reference tRNA ligase.

Suitable reference sequences include the Solanum melongena (eggplant) tRNA ligase which is shown in SEQ ID NO: 5.

Amino acid and nucleic acid sequence identity is generally defined with reference to the algorithm GAP (GCG Wisconsin Package™, Accelrys, San Diego Calif.). GAP uses the Needleman & Wunsch algorithm (J. Mol. Biol. (48): 444-453 (1970)) to align two complete sequences that maximizes the number of matches and minimizes the number of gaps. Generally, the default parameters are used, with a gap creation penalty=12 and gap extension penalty=4. Use of GAP may be preferred but other algorithms may be used, e.g. BLAST or TBLASTN (which use the method of Altschul et al. (1990) J. Mol. Biol. 215: 405-410), NBLAST and XBLAST (Altschul et al., 1991, Nucleic Acids Res., 25:3389-3402), FASTA (which uses the method of Pearson and Lipman (1988) PNAS USA 85: 2444-2448), the Smith-Waterman algorithm (Smith and Waterman (1981) J. Mol Biol. 147: 195-197), Gapped BLAST, BLAST-2, WU-BLAST 2 (Altschul et al., 1996, Methods in Enzymology, 266:460-480), ALIGN, ALIGN-2 (Genentech, CA USA), Megalign (DNASTAR), and the Bestfit program (Wisconsin Sequence Analysis Package, Genetics Computer Group, WI USA 53711), generally employing default parameters.

Particular amino acid sequence variants may differ from a reference sequence by insertion, addition, substitution or deletion of 1 amino acid, 2, 3, 4, 5-10, 10-20 or 20-30 amino acids. Particular nucleotide sequence variants may differ from a reference sequence by insertion, addition, substitution or deletion of 1 base, 2, 3, 4, 5-10, 10-20 or 20-30 bases.

Sequence identity is preferably determined over the full length of the sequences being compared.

In some preferred embodiments, the plant viroid and the tRNA ligase may be from the same plant species. For example, eggplant tRNA ligase may be expressed with an ELVd scaffold or avocado tRNA ligase may be expressed with an Avocado sunblotch viroid (ASBVd) scaffold.

The chimeric RNA molecule and the tRNA ligase are encoded by isolated nucleic acids in the host cell. The nucleic acids are heterologous or exogenous to the host cell and may be introduced into the host cell by transformation or transfection as described below.

Another aspect of the invention provides an isolated nucleic acid encoding a chimeric RNA molecule comprising a target RNA and a plant viroid scaffold, as described above.

The nucleotide sequence encoding the target RNA may be inserted within the nucleotide sequence encoding the plant viroid scaffold, as described above.

Nucleic acids as described above may be comprised within an expression vector.

Suitable vectors can be chosen or constructed, containing appropriate control sequences, including promoter sequences, terminator fragments, polyadenylation sequences, enhancer sequences, marker genes and other sequences as appropriate. Preferably, the vector contains appropriate regulatory sequences to drive the expression of the nucleic acid in a host cell, as described above. An example of a suitable vector for expression of a chimeric RNA molecule is shown in SEQ ID NOs: 6-8.

Suitable control sequences to drive the expression of heterologous nucleic acid coding sequences in expression systems are well-known in the art and include constitutive promoters, for example viral promoters such as CMV or SV40, and inducible promoters, such as Tet-on controlled promoters. For example, the tRNA ligase may be constitutively expressed in the host cell. A vector may also comprise sequences, such as origins of replication and selectable markers, which allow for its selection and replication and expression in bacterial hosts such as E. coli.

In some embodiments, a phage RNA polymerase promoter may be operably linked to the inserted nucleic acid and the chimeric RNA molecule obtained by in vitro transcription. For example, the vector may be incubated with ribonucleotide triphosphates, buffers, magnesium ions, and an appropriate phage RNA polymerase, such as SP6, T7 and T3 polymerase, under conditions for transcription of chimeric RNA molecules from the coding sequence. Suitable techniques are well-known in the art and appropriate reagents are commercially available (e.g. Applied Biosystems/Ambion, TX USA).

Another aspect of the invention provides a vector comprising a nucleic acid encoding a chimeric RNA molecule, as described above.

An example of a suitable vector for expression of a chimeric RNA molecule is shown in SEQ ID NOS: 7 and 8.

The vector may further comprise the nucleic acid encoding the tRNA ligase or the nucleic acid encoding the tRNA ligase may be contained in in a separate vector.

Another aspect of the invention provides a pair of vectors, the first vector comprising a nucleic acid encoding a chimeric RNA molecule as described above and the second vector comprising a nucleic acid encoding a tRNA ligase, as described above.

Vectors may be plasmids, viral e.g. phage, or phagemid, as appropriate. For further details see, for example, Molecular Cloning: a Laboratory Manual: 3rd edition, Russell et al., 2001, Cold Spring Harbor Laboratory Press. Many techniques and protocols for manipulation of nucleic acid, for example in preparation of nucleic acid constructs, mutagenesis, sequencing, introduction of DNA into cells and gene expression, are known in the art (see for example Protocols in Molecular Biology, Second Edition, Ausubel et al. eds. John Wiley & Sons, 1992; Recombinant Gene Expression Protocols Ed RS Tuan (March 1997) Humana Press Inc).

Nucleic acids or vectors as described above may be introduced into a host cell for example by transfection or transformation.

Techniques for the introduction of nucleic acid into cells are well established in the art and any suitable technique may be employed, in accordance with the particular circumstances. For eukaryotic cells, suitable techniques may include calcium phosphate transfection, DEAE-Dextran, electroporation, liposome-mediated transfection and transduction using retrovirus or other virus, e.g. adenovirus, AAV, lentivirus or vaccinia. For bacterial cells, suitable techniques may include calcium chloride transformation, electroporation and transfection using bacteriophage.

Marker genes such as antibiotic resistance or sensitivity genes may be used in identifying clones containing nucleic acid of interest, as is well-known in the art.

The introduced nucleic acid(s) may be on an extra-chromosomal vector within the cell or the nucleic acid may be integrated into the genome of the host cell. Integration may be promoted by inclusion of sequences within the nucleic acid or vector which promote recombination with the genome, in accordance with standard techniques.

In some embodiments, the host cells may express an exogenous tRNA ligase. For example, the host cells may have been previously transformed with nucleic acid encoding the tRNA ligase. A method as described herein may comprise introducing nucleic acid encoding the chimeric RNA molecule into host cells that express an exogenous tRNA ligase.

The introduction may be followed by expression of the nucleic acid(s) to produce the encoded chimeric RNA molecule and/or tRNA ligase. In some embodiments, host cells (which may include cells actually transformed although more likely the cells will be descendants of the transformed cells) may be cultured in vitro under conditions for expression of the nucleic acid, so that the encoded chimeric RNA molecule and/or tRNA ligase is produced. When an inducible promoter is used, expression may require the activation of the inducible promoter. The chimeric RNA molecules may then be recovered from the host cells or the surrounding medium.

Another aspect of the invention provides a recombinant host cell that expresses a chimeric RNA molecule and tRNA ligase, as described herein.

The host cell may comprise a heterologous nucleic acid encoding the tRNA ligase and a heterologous nucleic acid encoding the chimeric RNA molecule.

The nucleic acids may be contained in the same or separate expression vectors as described above.

Another aspect of the invention provides a recombinant host cell that expresses an exogenous tRNA ligase and is suitable for transformation with a nucleic acid encoding a chimeric RNA molecule, as described herein.

Host cells, chimeric RNA molecules and tRNA ligases are described in more detail above.

The expressed chimeric RNA molecule comprising the target RNA may be isolated and/or purified, after production from the host cell and/or culture medium. This may be achieved using any convenient method known in the art. Techniques for the purification of recombinant polypeptides are well known in the art and include, for example HPLC, FPLC or affinity chromatography. For example, chimeric RNA molecules may be purified to homogeneity using chromatography and/or electrophoresis, as described herein.

The chimeric RNA molecule produced in the host cells is preferably monomeric.

The chimeric RNA molecule produced in the host cells may be linear or circular. In some embodiments, the chimeric RNA molecules produced in a host cell may be a mixture of linear and circular molecules.

In some preferred embodiments, circular chimeric RNA molecules may be purified or isolated from the host cells. For example, circular RNA molecules may be more resistant to RNases than linear RNA molecules and may be more easily separated from other cellular RNAs by standard separation techniques, since host cells, such as E. coli, may lack endogenous circular RNAs.

Following isolation and/or purification, the chimeric RNA molecule may subsequently be used as desired, e.g. in the formulation of a composition which may include one or more additional components, such as a pharmaceutical composition which includes one or more pharmaceutically acceptable excipients, vehicles or carriers (e.g. see below).

In some embodiments, the target RNA may be extracted from the chimeric RNA molecule, for example by cleavage from the plant viroid scaffold.

Suitable techniques for separating the target RNA from the plant viroid scaffold are well known in the art (see for example; Batey R. T. (2014) Curr. Opin. Struct. Biol. 26C: 1-8).

In other embodiments, extraction of the target RNA from the chimeric RNA molecule may be unnecessary.

In some embodiments, the target RNA or chimeric RNA molecule may be modified or adapted after isolated from the host cell. For example, the 2′-OH group of one or more nucleotides in the target RNA or chimeric RNA molecule may be chemically modified, for example by addition of a substituent group, such as methyl (e.g. 2′-O-methyl), halogen (e.g. 2′-Fluoro) or amine (e.g. 2′-NH₃), or reduction to 2′-H (e.g. 2′deoxy).

Target RNAs and chimeric RNA molecules produced as described may be investigated further, for example the pharmacological properties and/or activity may be determined. Methods and means of RNA analysis are well-known in the art.

Another aspect of the invention provides a system for the production of RNA comprising;
a host cell,
a nucleic acid encoding a chimeric RNA molecule comprising a target RNA and a plant viroid scaffold, and
a nucleic acid encoding a tRNA ligase.

Expression of the nucleic acids in the host cell leads to the production of large amounts of chimeric RNA molecule comprising the target RNA, as described above. For example, more than 10 mg, more than 20 mg, more than 30 mg, more than 50 mg or more than 100 mg of monomeric chimeric RNA may be produced per litre of the host cell culture. The data herein, for example, shows the production of 150 mg ELVd RNA, 75 mg ELVd-Spinach RNA and 30 mg ELVdI3D1-Spinach RNA per litre of E. coli culture.

Suitable host cells and nucleic acids are described above.

Another aspect of the invention provides a kit for the production of RNA comprising;
a nucleic acid encoding a chimeric RNA molecule comprising a target RNA and a plant viroid scaffold; or
a nucleic acid encoding a plant viroid scaffold,
   said nucleic acid comprising a cloning site for insertion of a heterologous nucleotide sequence encoding a target RNA.

Cloning of the heterologous nucleotide sequence into the cloning site introduces the target RNA into the plant viroid scaffold.

In some embodiments, the kit may further comprise a nucleic acid encoding a tRNA ligase, for example in an expression vector.

In other embodiments, the kit may further comprise a host cell that expresses a heterologous nucleic acid encoding a tRNA ligase.

Suitable nucleic acids, vectors and host cells are described above.

The kit may include instructions for use in a method of production of RNA as described above.

A kit may include one or more other reagents required for the method, such as buffer solutions and DNA and/or RNA isolation and purification reagents.

A kit may include one or more articles for performance of the method, such as means for providing the test sample itself, including sample handling containers (such components generally being sterile).

Various further aspects and embodiments of the present invention will be apparent to those skilled in the art in view of the present disclosure.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Certain aspects and embodiments of the invention will now be illustrated by way of example and with reference to the figures described above.

### EXPERIMENTS

### 1. Materials and Methods

### 1.1 Plasmid Construction

Plasmids were constructed using standard molecular biology techniques and assembly using IIS restriction enzymes and T4 DNA ligase (Engler and Marillonnet, 2014). PCR amplifications were performed with the Phusion High-Fidelity DNA polymerase (Thermo Scientific) in HF buffer (Thermo Scientific), 3% dimethyl sulfoxide and 0.2 mm each NTPs. Plasmid sequences were confirmed by sequencing (3130xl Genetic Analyzer; Life Technologies).

### 1.2 E. Coli Cultures

E. coli BL21(DE3) (Novagen) were electroporated or co-electroporated (ECM 399, BTX) with the different plasmids and recombinant clones selected by growing overnight at 37° C. in LB solid medium (10 g/l tryptone, 5 g/l yeast extract, 10 g/l NaCl, 1.5% agar). Selection of recombinant clones was done using the antibiotics ampicillin (50 μg/ml), chloramphenicol (34 μg/ml) or both. Liquid cultures were carried out in LB (as above but without agar) or TB (12 g/l tryptone, 24 g/l yeast extract, 0.4% glycerol, 0.17 M KH₂PO₄ and 0.72 M K₂HPO₄) liquid media, containing the appropriate antibiotics, at the indicated temperature (in general 37° C.) with vigorous shaking (225 revolutions per min—rpm—). Cell densities were measured at 600 nm with a colorimeter (CO8000, WPA). Induction of protein expression was carried out by adding the appropriate amount of 0.25 M IPTG to the culture.

### 1.3 RNA Extraction and Analysis

For analytical purposes, aliquots of 2 ml of cultures were taken at the desired time points. Cells were sedimented by centrifuging at 13,000 rpm for 2 min and, in general, resuspended in 50 μl of TE buffer (40 fold concentration) by vortexing. One volume (50 μl) of a 1:1 (v/v) mix of phenol (saturated with water and equilibrated at pH 8.0 with Tris-HCl, pH 8.0) and chloroform was added and the cells broken by vigorous vortexing. Phases were separated by centrifuging for 5 min at 13,000 rpm. The aqueous phases were recovered and re-extracted with 1 volume (50 μl) of chloroform, vortexing and centrifuging in the same conditions. The aqueous phases containing total bacterial nucleic acids were finally recovered by pipetting and either subjected directly to further analysis or stored frozen at −20° C.

Total RNA was analyzed by denaturing PAGE. In general, 20 μl of RNA preparations were mixed with 1 volume (20 μl) of loading buffer (98% formamide, 10 mM Tris-HCl, pH 8.0, 1 mM EDTA, 0.0025% bromophenol blue and 0.0025% xylene cyanol), heated for 1.5 min at 95° C. and snap cooled on ice. PAGE separation was done for 2:30 h at 200 V in 5% polyacrylamide gels (37.5:1 acrylamyde:N,N′-methylenebisacrylamide) of 140×130×2 mm in TBE buffer (89 mM Tris, 89 mM boric acid, 2 mM EDTA) including 8 M urea (electrophoresis buffer was TBE without urea). Gels were stained by shaking for 15 min in 200 ml of 1 μg/ml ethidium bromide. After washing three times with water, the gels were photographed under UV light (UVIdoc-HD2/20MX, UVITEC). In some cases, separated RNAs were subjected to a second electrophoretic dimension. Lanes from 5% polyacrylamide, 8 M urea, TBE gels were cut and laid transversally on top of similar gels (5% polyacrylamide, 8 M urea) but containing 025×TBE buffer. These gels were run for 2:30 h at 350 V (25 mA maximum) and stained as described.

For northern blot hybridization analysis, RNAs separated by electrophoresis were electroblotted to positively charged nylon membranes (Nytran SPC; Whatman) and cross-linked by irradiation with 1.2 J/cm² UV light (254 nm; Vilber Lourmat). Hybridization was performed overnight at 70° C. in 50% formamide, 0.1% Ficoll, 0.1% polyvinylpyrrolidone, 100 ng/ml salmon sperm DNA, 1% SDS, 0.75 M NaCl, 75 mM sodium citrate, pH 7.0, with approximately 1 million counts per minute of ³²P-labelled ELVd RNA of complementary polarity. Hybridized membranes were washed three times for 10 min with 2×SSC, 0.1% SDS at room temperature and once for 15 min at 55° C. with 0.1×SSC, 0.1% SDS (1×SSC is 150 mM NaCl, 15 mM sodium citrate, pH 7.0). Results were registered by autoradiography using X-ray films (Fujifilm). The radioactive probe was produced by in vitro transcription of a linearized plasmid containing a dimeric ELVd (AJ536613) cDNA in the proper orientation. One μg of linearized plasmid (Hind III) was transcribed with 50 U of T3 bacteriophage RNA polymerase (Epicentre) in a 20-μl reaction containing 40 mM Tris-HCl, pH 8.0, 6 mM MgCl₂, 20 mM DTT, 2 mM spermidine, 0.5 mM each of ATP, CTP, and GTP, and 50 μCi of [α-³²P]UTP (800 Ci/mmol), 20 U RNase inhibitor (RiboLock; Thermo Scientific) and 0.1 U yeast inorganic pyrophosphatase (Thermo Scientific). Reactions were incubated for 1 h at 37° C. After transcription, the DNA template was digested with 20 U DNase I (Thermo Scientific) for 10 min at 37° C., and the probe was purified by chromatography using a Sephadex G-50 column (Mini Quick Spin Column; Roche Applied Science).

### 1.4 ELVd RNA Purification

For preparative purposes, 1 l of total E. coli culture in TB liquid medium distributed in four 1 l baffled Erlenmeyer flasks was grown at 37° C. with intense shaking (180 rpm). Protein expression was induced at DO₆₀₀=0.1 by adding IPTG at 0.1 mM. Cells were recovered at 14 h post-induction by centrifuging at 10,000 rpm for 10 min. Sedimented cells were resuspended in water and pelleted again in the same conditions. Cells were resuspended in 50 ml of chromatography buffer (50 mM Tris-HCl, pH 6.5, 0.15 M NaCl, 0.2 mM EDTA) and RNA extracted by adding 50 ml phenol:chloroform (1:1, pH 8.0) and vortexing. The aqueous phase, recovered by centrifuging 10 min at 10,000 rpm, was re-extracted with 50 ml of chloroform.

RNA in the second aqueous phase was filtered (0.2 μm; Filtropur S; Sarstedt) and chromatographed through a DEAE Sepharose column (HiTrap DEAE FF; GE Healthcare) of 5 ml at 5 ml/min using an ÄKTA Prime Plus liquid chromatography system (GE Healthcare). The column was equilibrated with 50 ml of chromatography buffer and the sample (35 ml at this point) loaded. The column was washed with 50 ml of chromatography buffer and the RNA eluted with 100 ml of chromatography buffer plus 1 M NaCl. Fractions (5 ml) were collected during elution and analyzed by denaturing PAGE.

Chromatography fraction 2 (RNA elution peak) was mixed with 1 volume of formamide loading buffer (see above) and the RNA denatured (see above). RNA was separated by two consecutive electrophoreses. First by PAGE in a 5% polyacrylamide, 8 M urea, TBE gel, as described above. After staining with ethidium bromide, the band of the gel containing the monomeric circular ELVd RNA was cut and loaded on top of a second gel (5% polyacrylamide—39:1 acrylamyde:N,N′-bis(acryloyl)cystamine—, TAE—40 mM Tris, 20 mM sodium acetate, 1 mM EDTA, pH 7.2—). After staining, the band of gel containing the monomeric circular ELVd RNA was cut and solubilized by adding 0.1 volumes of 2-mercaptoethanol. RNA was purified from the solution by chromatography with a DEAE Sepharose column as explained above.

### 1.5 Fluorescence Assay

To assay the fluorescence of RNA aptamer Spinach, E. coli cultures expressing ELVd-Spinach RNA were supplemented with 200 μM DFHBI and grown for 1 additional h. Pelleted bacteria were photographed under a stereomicroscope (Leica MZ 16 F) with UV illumination and a GFP2 filter (Leica). RNA extracts were supplemented with 20 μM DEHBI and photographed under the same conditions

### 2. Results

### 2.1 Co-Expression of an ELVd RNA and Eggplant tRNA Ligase in E. Coli

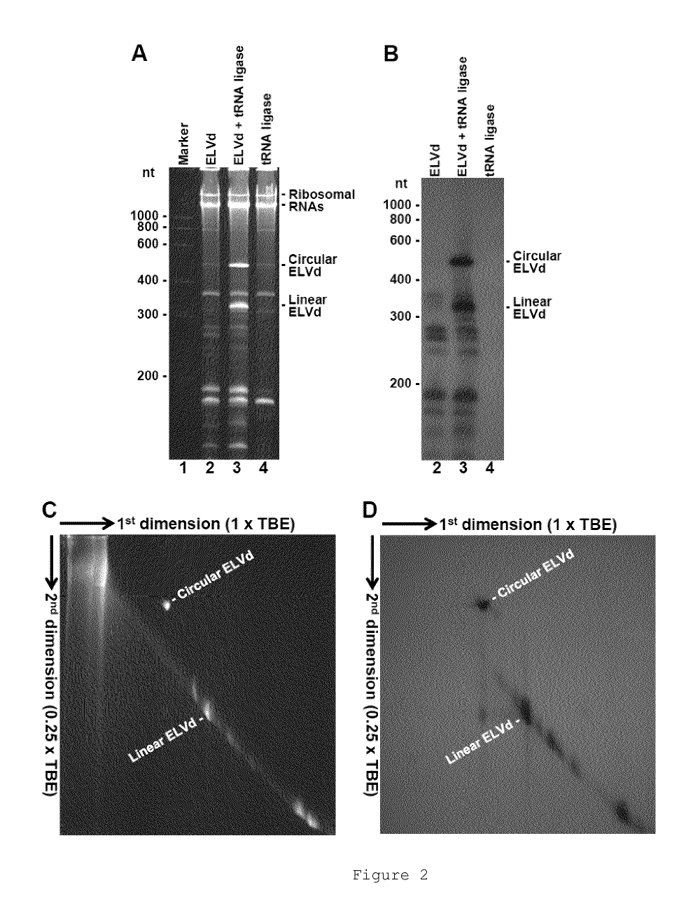

Recombinant eggplant tRNA ligase (GenBank accession number AFK76482) efficiently circularizes in vitro the monomeric linear ELVd RNA (333 nt; GenBank accession number AJ536613; FIG. 1) arising from ribozyme self-cleavage of a longer-than-unit transcript (Nohales et al., 2012b). To research the domains and residues of both the viroid RNA and the enzyme involved in recognition and catalysis, we set up an experimental system consisting of recombinant E. coli clones transformed with plasmids to co-express both molecules. A longer-than-unit ELVd RNA (386 nt; from C327 to G46; including the repetition of the plus-strand hammerhead ribozyme domain; note that ELVd RNA is circular and A333 is followed by G1) was expressed from plasmid pLELVd, with pUC replication origin and an ampicillin resistance gene, under the control of the E. coli murein lipoprotein promoter (GenBank accession number U00096.3, positions 1757293 to 1757385) and 5S rRNA (rrnC) terminator (GenBank accession number U00096.3, positions 3947033 to 3947076). The exact sequence of this plasmid is specified in SEQ ID NO: 6. A recombinant version of eggplant tRNA ligase, including its amino-terminal transit peptide mediating translocation to the chloroplasts (Englert et al., 2007), and a carboxy-terminal His₆ tail (Nohales et al., 2012b), was expressed from plasmid p15RnlSm. In this plasmid, which includes a p15A replication origin and a chloramphenicol resistance gene, tRNA ligase expression is under the control of T7 bacteriophage promoter and terminator. E. coli, strain BL21(DE3), expressing T7 bacteriophage RNA polymerase under the control of the isopropyl β-D-1-thiogalactopyranoside (IPTG)-inducible lacUV5 promoter, was transformed with either pLELVd, p15tRnlSm or both. Bacterial recombinant clones were grown at 25° C. to an optic density at 600 nm (OD₆₀₀) of 0.6 and induced with 0.4 mM IPTG. Growing was continued for 10 h and cells harvested by centrifugation. After resuspending the cells in TE buffer (10 mM Tris-HCl, pH 8.0, 1 mM ethylenediaminetetraacetic acid—EDTA—), total RNA from bacteria was extracted by breaking the cells with one volume of a phenol:chloroform (1:1; pH 8.0) and vortexing vigorously. Aliquots of the RNA preparations were separated by denaturing polyacrylamide gel electrophoresis (PAGE) in the presence of 8 M urea. The gel was stained with ethidium bromide and photographed under ultraviolet (UV) light (FIG. 2A). RNAs in the gel were electroblotted to a nylon membrane and analyzed by northern blot hybridization with a radioactive ELVd RNA probe of complementary polarity (FIG. 2B). This analysis showed that bacteria transformed with pLELVd accumulated several species of ELVd RNAs, although the expected processing product of the primary transcript (monomeric linear ELVd RNA) was not prominent (FIGS. 2A and B, lane 2). Bacteria co-transformed with pLELVd and p15tRnlSm accumulated similar amounts of the same ELVd species; but in contrast, they accumulated two additional prominent ELVd species whose position in the gel suggested they were the monomeric linear and circular ELVd RNAs (FIGS. 2A and B, lane 3). Interestingly, the accumulation level of these two ELVd species was similar to that of E. coli ribosomal RNAs (FIG. 2A, lane 3). Logically, bacteria transformed with p15tRnlSm did not accumulate any ELVd RNA (FIGS. 2A and B, lane 4). Analysis of RNA from bacteria co-transformed with pLELVd and p15tRnlSm by two dimensional PAGE, followed by northern blot hybridization, confirmed that the two prominent species were, indeed, monomeric linear and circular ELVd RNAs (FIGS. 2C and D). The absence of oligomeric ELVd RNA species, typical of the viroid rolling-circle replication mechanism, neglected the possibility of ELVd replication in these recombinant E. coli cells (FIG. 2).

These results suggested that in our experimental conditions, when a longer-than-unit ELVd transcript is expressed in E. coli, it undergoes degradation and only low amounts of small sized ELVd RNAs can be detected by northern blot hybridization. However, the co-expression of eggplant tRNA ligase leads to strong accumulation of ELVd processing products. An ELVd transcript, similar to that assayed in this experiment, containing a repeated hammerhead ribozyme domain is able to efficiently self-cleave in vitro to monomeric length (Fadda et al., 2003). Self-cleavage is also efficient when these kind of transcripts are expressed in vivo in Chlamydomonas reinhardtii chloroplasts (Martinez et al., 2009). Consequently, our results show that, in E. coli, co-expressed eggplant tRNA ligase circularizes part of the monomeric linear ELVd RNA produced by the hammerhead ribozymes, and most important, contributes somehow to a remarkable intracellular stability of both processing products, monomeric linear and circular ELVd RNAs.

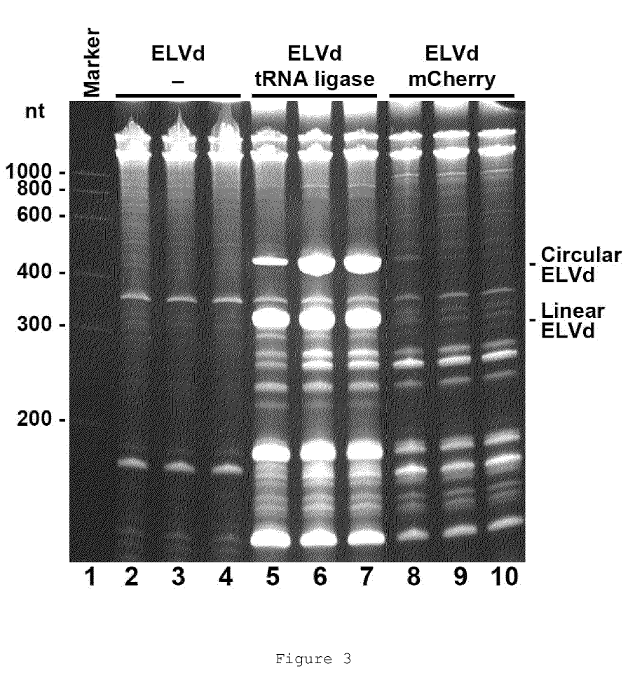

To determine whether the strong accumulation of ELVd RNA in E. coli was specifically driven by eggplant tRNA ligase or any recombinant protein overexpressed in E. coli could induce the same effect, we created plasmid p15mCherry, in which the cDNA coding for the red fluorescent protein mCherry (GenBank accession number AY678264) (Shaner et al., 2004) replaced the cDNA encoding the tRNA ligase in p15tRnlSm. We co-transformed E. coli with pLELVd and either p15tRnlSm or p15mCherry, grow independent recombinant clones at 37° C. and induced expression of both recombinant proteins. Analysis of total RNA extracted from cells harvested 8 h after induction showed that monomeric linear and circular ELVd RNAs only accumulated to remarkable levels in cells co-expressing tRNA ligase (FIG. 3, compare lanes 5 to 7 with lanes 9 to 10). Efficient expression of mCherry was corroborated by the strong red color of the corresponding recombinant E. coli clones.

### 2.2 Overproduction of Circular ELVd RNA in E. Coli

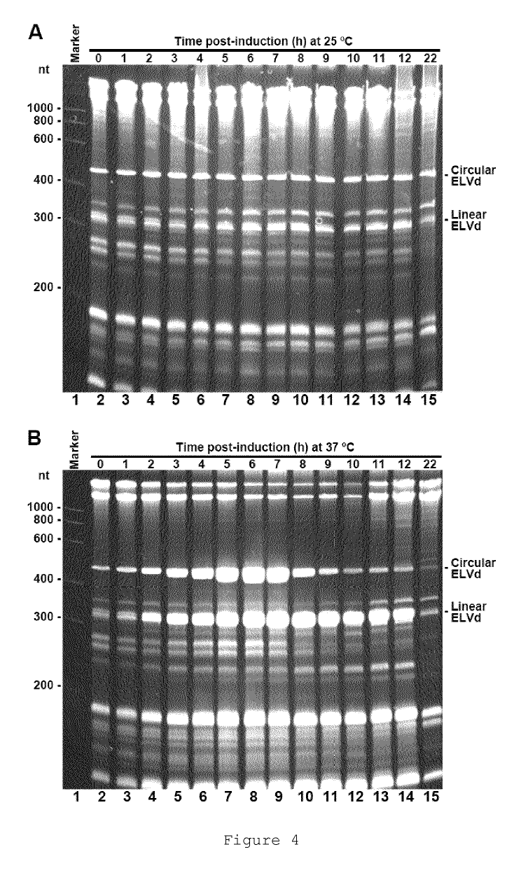

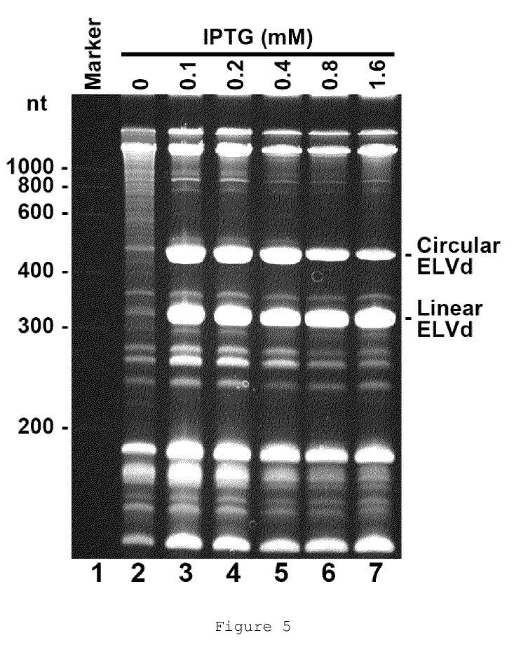

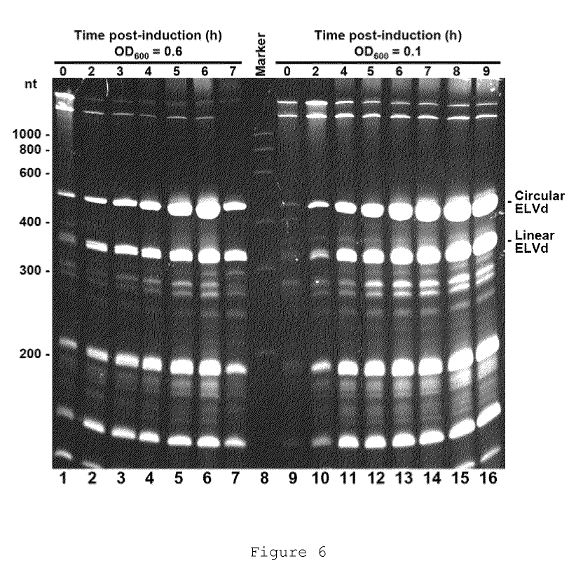

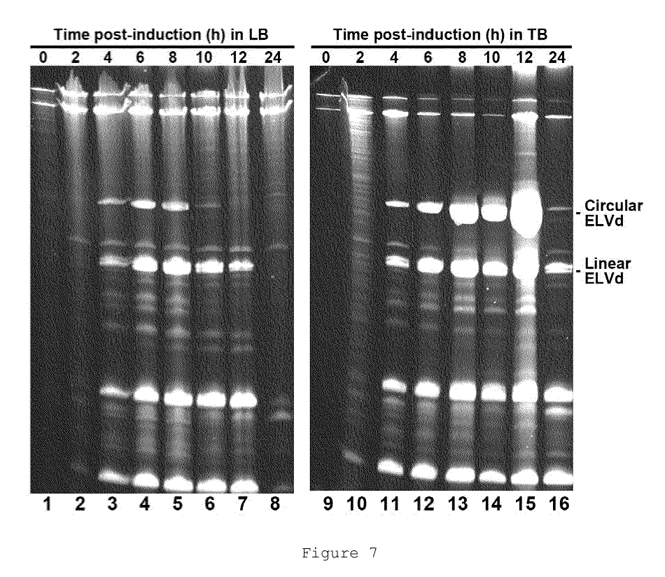

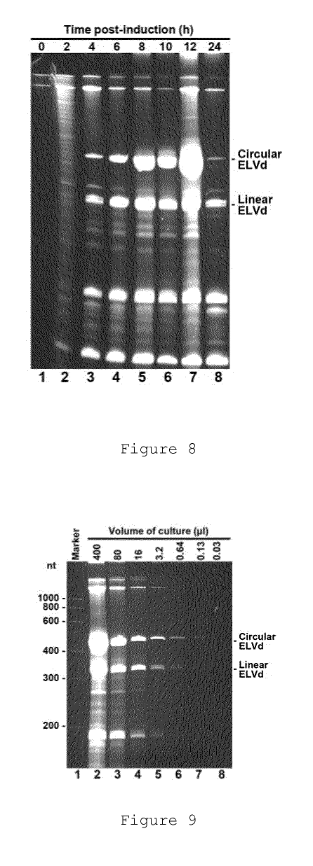

The results showed that E. coli could be a good biofactory to produce substantial amounts of ELVd circular RNA. In contrast to that accumulating in an infected plant, this RNA is the product of direct transcription and processing, not replication, and it should be genetically more homogenous, which is an advantage for some studies. So, we tried to optimize the E. coli growing and the eggplant tRNA ligase induction conditions to maximize ELVd RNA accumulation. We grow cultures of E. coli co-transformed with pLELVd and p15tRnlSm in different conditions, took aliquots of the cultures at several time points, extracted total RNA from the cells, and analyzed them by denaturing PAGE and ethidium bromide staining of the gels. Comparison of two time-course expressions at 25 and 37° C. indicated that ELVd accumulation in E. coli was higher at 37° C. (FIG. 4). Interestingly, the time course analysis at 37° C. showed how ELVd accumulation goes up and down with time at this temperature (FIG. 4B). Parallel expressions in which tRNA ligase was induced with different amounts of IPTG indicated that ELVd accumulation was better at the lowest assayed IPTG concentration (0.1 mM) (FIG. 5). Remarkably, this experiment corroborated that tRNA ligase plays a crucial role in the system, because negligible amounts of ELVd accumulated in non-induced controls (FIG. 5, compare, lane 2 with 3 to 7). Analysis of two time-course expressions in which IPTG to 0.1 mM was added at cell density of OD₆₀₀=0.6 or OD₆₀₀=0.1 showed better performance in the second case (FIG. 6). A comparison of the classical Luria-Bertani (LB) medium with the richest Terrific Broth (TB) indicated that ELVd accumulation was much higher in cells grown in the second medium (FIG. 7). Finally, we conducted a time-course analysis in our best conditions to produce ELVd RNA. A culture was grown in TB medium at 37° C. and tRNA ligase expression induced at cell density of OD₆₀₀=0.1 with 0.1 mM IPTG. Aliquots were taken at different time points and total bacterial RNA analyzed as described. The amount of monomeric linear and circular ELVd RNAs per volume of culture increased with time reaching a maximum around 12 h post-induction and then decreased to essentially disappear at 24 h (FIG. 8).

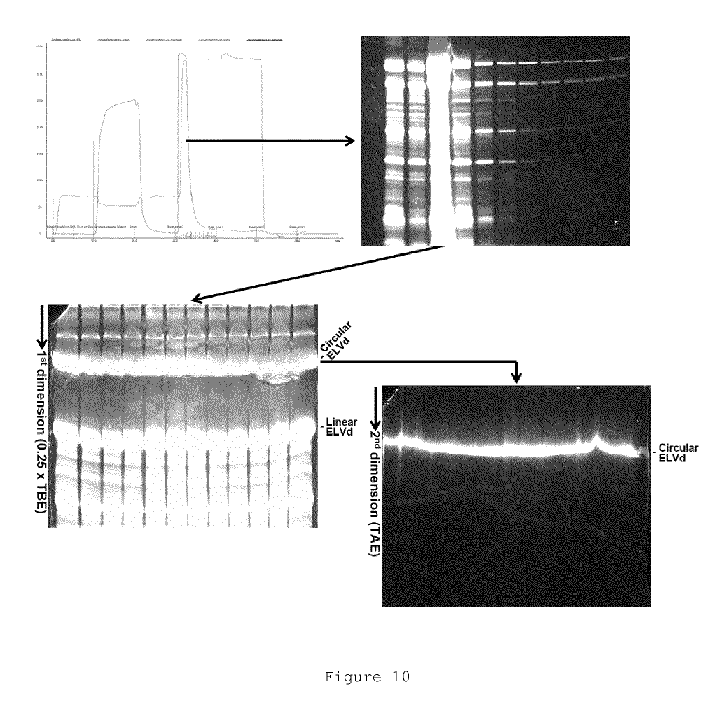

By serially diluting the RNA preparation from the aliquot taken at 12 h after induction, analyzing by denaturing PAGE and ethidium bromide staining, and comparing with RNA standards of known concentration, we calculated an accumulation of approximately 150 mg of monomeric linear and circular ELVd RNAs per liter of culture at this time point (FIG. 9). This analysis also showed that, following our RNA extraction procedure, approximately half of the ELVd monomeric RNA was recovered in a circular form and half in a linear form. FIG. 10 shows a pilot expression and purification process in which circular ELVd RNA was produced in E. coli, extracted from the cells by phenol:chloroform treatment, purified by anion exchange chromatography using a diethylaminoethyl (DEAE) Sepharose column and further purified to homogeneity by two sequential PAGEs, first under denaturing conditions and then under native conditions. The second gel was cross-linked with N,N′-bis(acryloyl)cystamine that allows solubilization of the band by a treatment with 2-mercaptoethanol. Taken together, these results confirm that E. coli is a good platform to overproduce large amounts of ELVd RNA.

### 2.3 Overproduction of Recombinant RNAs in E. Coli Using ELVd as a Scaffold

Then, we wondered whether an RNA of interest, a recombinant RNA, could be inserted in the ELVd molecule and overproduced in our E. coli system. We chose as insertion position in the RNA molecule the terminal loop of the upper-right hairpin present in the theoretical ELVd conformation of minimum free energy (FIG. 1). We chose this position because, our previous mutational analysis of the viroid molecule demonstrated that an insertion of eight nucleotides (GCGGCCGC) in this particular position does not abolish viroid infectivity in eggplant, in contrast to other assayed positions (Martinez et al., 2009). As a recombinant RNA, we chose the RNA aptamer Spinach, a 98-nt-long RNA that when binding to the fluorophore 3′5-difluoro-4-hydroxybenzylidene (DFHBI) emits a green fluorescence comparable in brightness with fluorescent proteins (Paige et al., 2011).

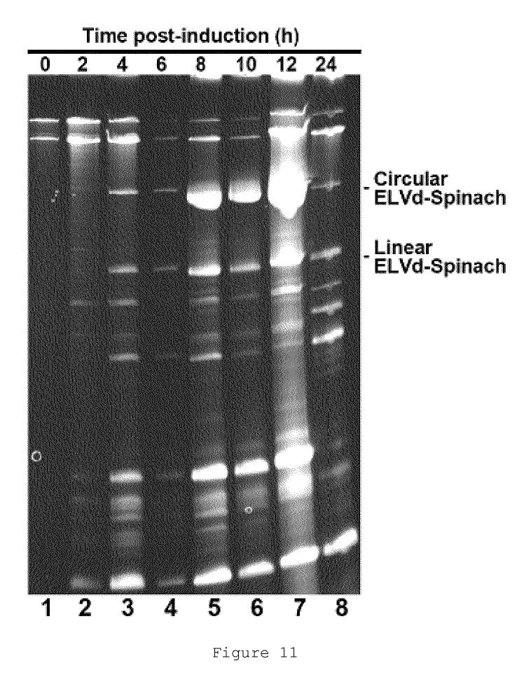

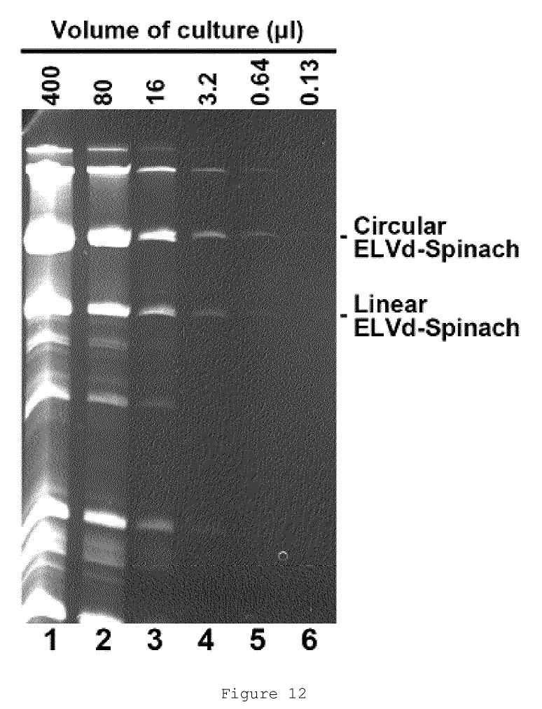

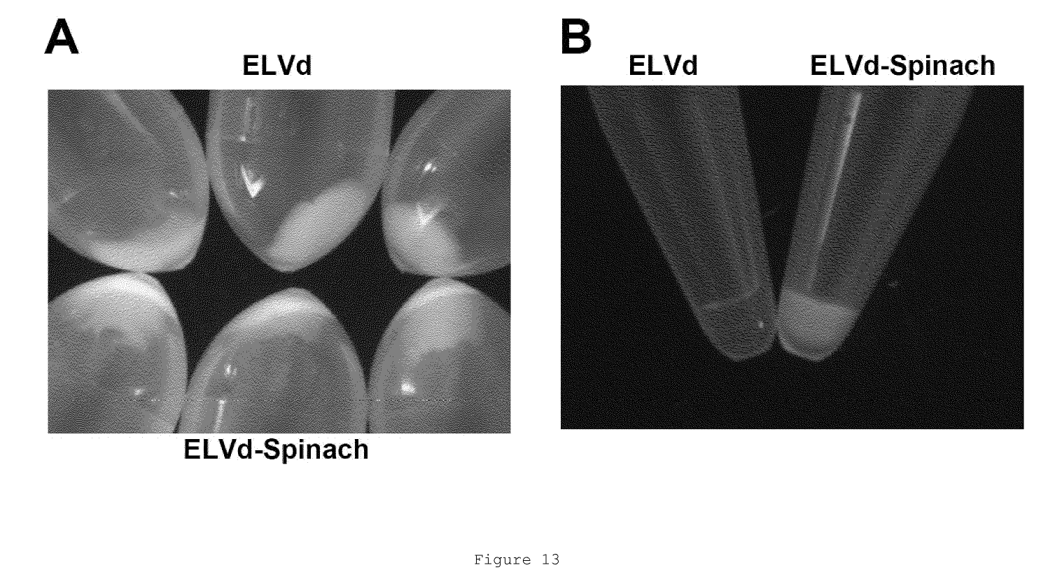

We constructed plasmid pLELVd-Spinach (SEQ ID NO: 7) in which a cDNA coding for Spinach (Paige et al., 2011) was inserted between positions T245-T246 of the cDNA coding for ELVd in pLELVd. E. coli BL21 (DE3) was co-transformed with pLELVd-Spinach and p15tRnlSm. Recombinant clones were grown and induced in our optimized conditions (37° C. in TB medium; induction at OD₆₀₀=0.1 with 0.1 mM IPTG) and aliquots of the cultures taken at different time points. RNA was extracted from the bacterial cells present in the aliquots and analyzed by denaturing PAGE, followed by ethidium bromide staining of the gel (FIG. 11). Interestingly, the ELVd-Spinach chimeric RNA also accumulated in the recombinant E. coli cells to remarkable amounts. A PAGE analysis of serial dilutions of the RNA preparation obtained from the aliquot showing maximum accumulation indicated a production of approximately 75 mg of ELVd-Spinach RNA per liter of bacterial culture (FIG. 12). Again, the recombinant RNA was recovered half and half in circular and linear forms (FIG. 12). As the functionality of Spinach can be tested both in vitro and in vivo (Paige et al., 2011), we added DFHBI to both our E. coli culture and RNA preparation. In both cases we observed emission of strong and bright green fluoresce (FIG. 13), showing the functionality of the Spinach aptamer produced in our E. coli system and embedded in the ELVd molecule. These results demonstrate that our E. coli system can be a biofactory to produce large amounts of recombinant RNAs and that these RNAs can be properly folded to perform their functions.

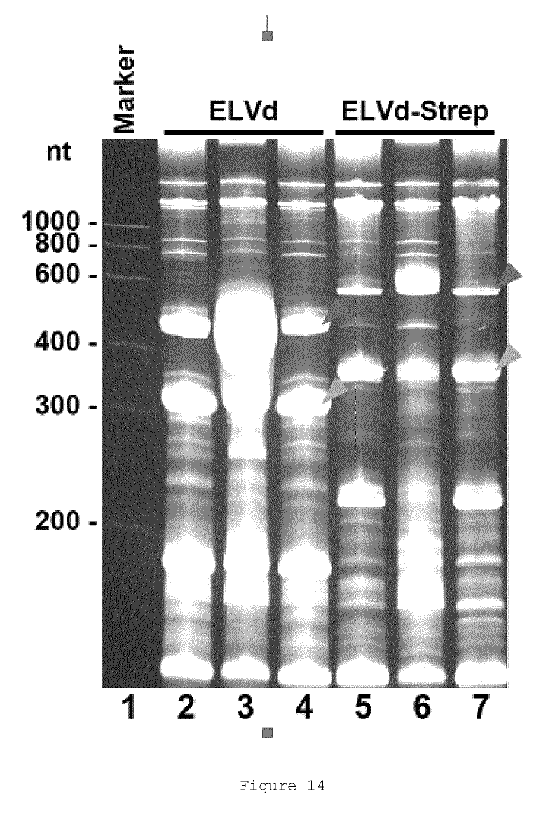

To prove that the case of Spinach was not an exception and that our system to produce recombinant RNA in E. coli could be applied in a more general manner, we tried to produce another RNA aptamer. This time we chose the 42-nt-long streptavidin binding aptamer (Srisawat and Engelke, 2001; Srisawat and Engelke, 2002). We constructed plasmid pLELVd-Strep (SEQ ID NO: 8), in which a cDNA coding for the streptavidin-binding aptamer was inserted between positions T245-T246 of the cDNA coding for ELVd in pLELVd. Again, co-transformed E. coli (pLELVd-Strep and p15tRnlSm) showed a remarkable accumulation of the hybrid molecule (ELVd-Strep) in both linear and circular forms (FIG. 14) Chromatographic fractionation of the RNA preparation demonstrated the binding of the hybrid RNA to a streptavidin Sepharose column.

### 2.4 Optimization of the E. Coli System to Overproduce Recombinant RNA

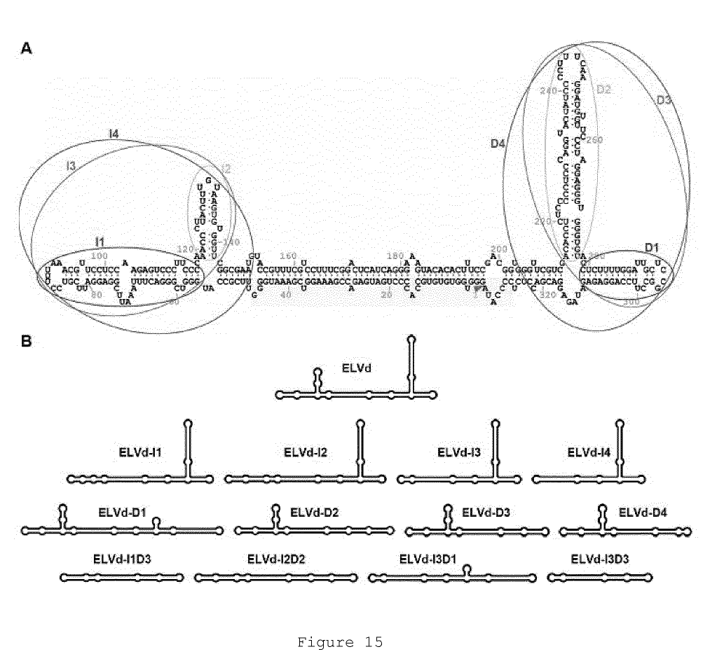

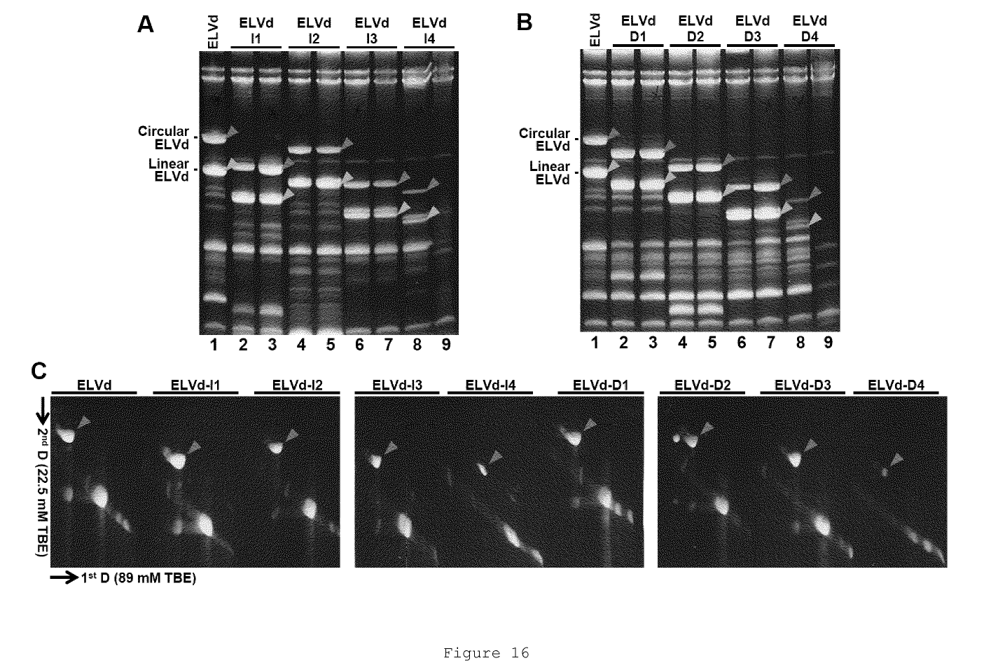

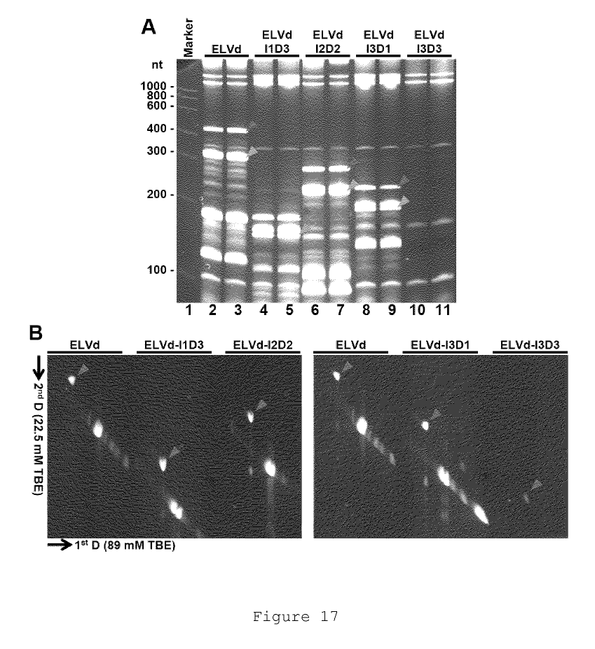

We seek the optimization of the E. coli system to overproduce RNA aptamers. First, we investigated the possibility of reducing the size of the ELVd scaffold in the final product. For this, we prepared a series of plasmids derived from pLELVd in which different fragments of the viroid cDNA were deleted (FIG. 15). We analyzed how deletions affected accumulation of the resulting ELVd RNA in E. coli. The rational of the deletion scheme was to sequentially remove elements of the viroid secondary structure of minimum free energy without affecting the hammerhead ribozyme domain, which mediates self-cleavage of the primary transcript (FIG. 15A). Initially, we assayed single deletions affecting the left (, I1 from 56 to 116; I2, from 119 to 142, I3 from 56 to 141, and I4 from 48 to 149) and the right (D1 from 279 to 310; D2, from 214 to 276; D3 from 214 to 310, and D4 from 205 to 324) sides of the molecule. I and D stand from the Spanish words (izquierda and derecha) for left and right, respectively. E. coli BL21(DE3) were transformed with p15tRnlSm and the corresponding pLELVd derivative plasmid. Several independent recombinant colonies bearing each deletion were grown and induced. RNAs were extracted from each culture at the same time point and analyzed by denaturing PAGE and ethidium bromide staining (FIGS. 16A and B). One of the samples for each deletion was further separated by PAGE in a second dimension to solve ambiguities about the circular nature of the accumulating RNA products (FIG. 16C). Most deletions did not affect accumulation of ELVd RNAs, except for ELVd-I4 and ELVd-D4 (FIG. 16). Then, we combined double deletions from the left and right sides of the molecule based on initial results. We created new plasmids with deletions ELVd-I1D3, -I2D2, -I3D1 and -I3D3. Analysis of RNAs accumulating in transformed E. coli indicated that only double deletion I3D3 drastically affected ELVd RNA accumulation (FIG. 17).

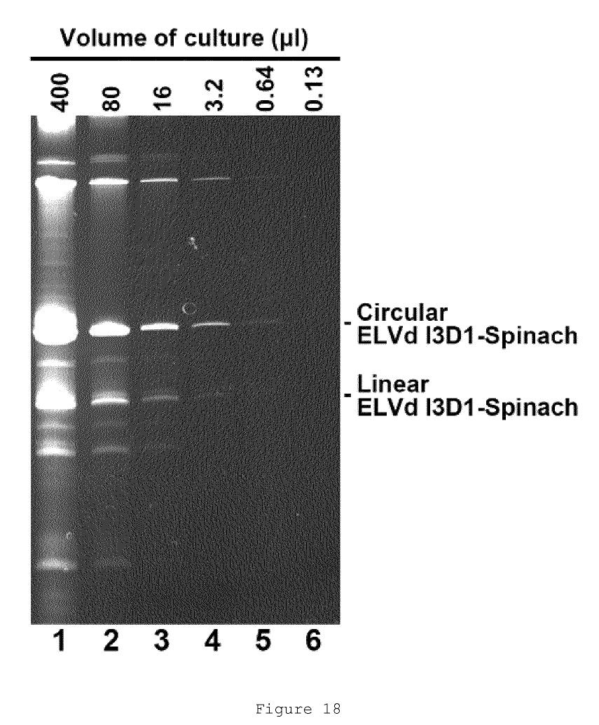

From this analysis two ELVd forms emerged as potential reduced scaffolds to produce recombinant RNA in E. coli, ELVd-I1D3 (175 nt) and ELVd-I3D1 (215 nt). We focused on deleted form ELVd-I3D1 because it maintains the upper-right hairpin where the Spinach and the streptavidin-binding aptamers were successfully inserted in full-length ELVd. We constructed a version of pLELVd-Spinach in which the I3D1 double-deletion was created (pLELVdI3D1-Spinach). When we transformed E. coli with this plasmid along with p15tRnlSm, grown and induced recombinant clones, and analyzed total RNA from the corresponding bacterial cells, we observed a large accumulation of the new ELVdI3D1-Spinach chimeric RNA in both linear and circular forms. A PAGE analysis of serial dilutions of the RNA preparation indicated an accumulation of approximately 30 mg recombinant ELVdI3D1-Spinach RNA per liter of E. coli culture (FIG. 18).

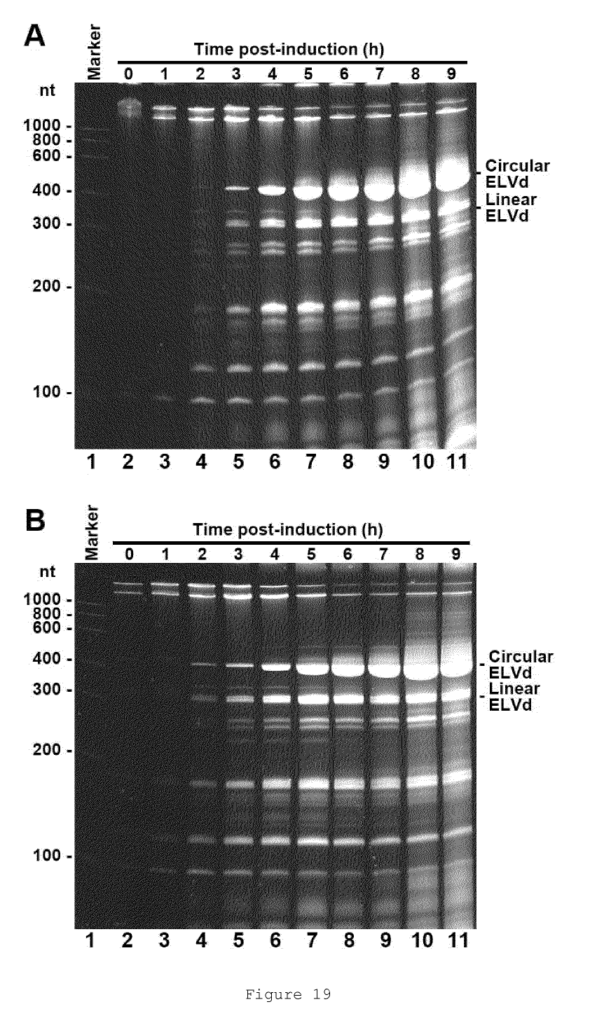

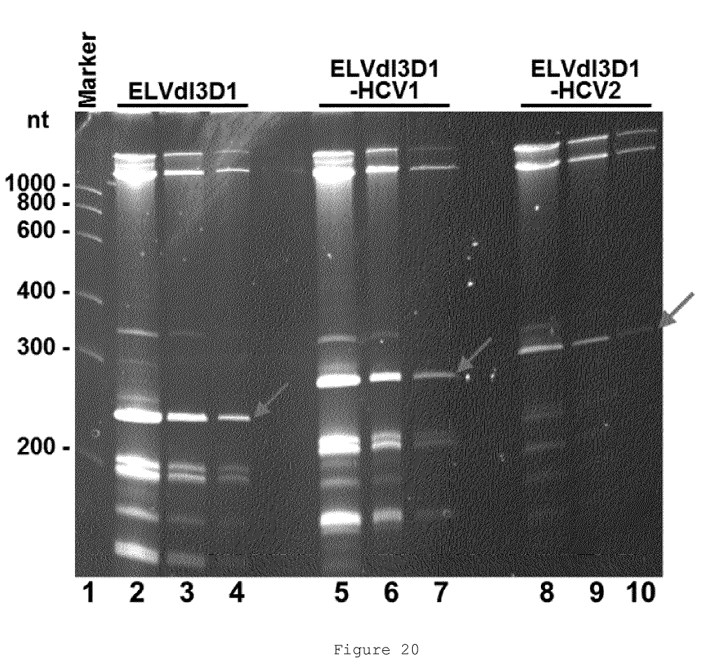

Second, we rationalized that the necessity of inducing eggplant tRNA ligase expression by adding IPTG to the culture could be an undesired step for future industrial applications of our system. For this reason, we constructed a new plasmid (p15LtRnlSm), derived from p15tRnlSm, in which the T7 bacteriophage RNA polymerase promoter was replaced by the E. coli murein lipoprotein constitutive promoter. This is the same promoter that drives expression of the ELVd RNA in pLELVd. E. coli BL21(DE3) were co-transformed with pLELVd-Spinach and either the original p15tRnlSm or the new p15LtRnlSm. The two E. coli recombinant clones were grown and at OD₆₀₀=0.1 induced or not with IPTG. A time-course analysis of the RNA in both bacterial clones showed no difference in recombinant RNA accumulation (FIG. 19). This result demonstrates that the inducible strategy initially used in our system to express eggplant tRNA ligase can be substituted by a constitutive approach without substantially affecting recombinant RNA production.

### 2.5 Overproduction of Recombinant RNAs in Other Expression Systems Using ELVd as a Scaffold

Recombinant RNA may be produced using plant viroids in bacterial species other than E. coli and eukaryotic expression systems, such as yeast (e.g. S. cerevisiae) and mammalian cells. For this purpose, specific plasmids appropriate to express RNAs and proteins in those systems are used. Using appropriate promoters, the viroid RNA scaffold (full-length or deleted) with the inserted RNA of interest is expressed along with the eggplant tRNA ligase. For example, in the case of the yeast S. cerevisiae, a conventional laboratory strain, such as BY4741 (MATa his3-Δ1 leu2-Δ0 met15-Δ0 ura3-Δ0), is transformed with a suitable expression plasmid, such as pFL61 (URA, 2μ, PGK1 promoter) (Minet, M et al 1992; Plant J. 2: 417-422) containing a construct to express ELVd RNA with duplicated hammerhead domain (386 nt; from C327 to G46; including the repetition of the plus-strand hammerhead ribozyme domain; note that ELVd RNA is circular and A333 is followed by G1) and the RNA of interest, such as the RNA aptamer Spinach (Paige et al., 2011) inserted, as explained above. Eggplant tRNA ligase (GenBank accession number AFK76482) is co-expressed, for example using yeast expression plasmids that allow Gateway cloning (Alberti et al., 2007; Yeast 24: 913-919). Viroid RNA is processed through hammerhead ribozymes, as in E. coli, and the resulting viroid RNA scaffold is recognized, circularized and bound by tRNA ligase. Co-expression of both elements of the system in S. cerevisiae and other cell types results in over-accumulation of an RNA-protein complex between the tRNA ligase and the chimeric RNA molecule. Chimeric RNA is extracted from the expressing cells by phenol:chloroform treatment and purified by chromatography and electrophoresis techniques as described above for E. coli. To extract RNA from yeast cells, mechanical disruption of cells using glass beads may also be used (Sasidharan K. et al Yeast 29: 311-322).

Other yeast species, including Arxula adeninivorans (Blastobotrys adeninivorans), Candida boidinii, Hansenula polymorphia (Pichia angusta), Kluyveromyces lactis, Pichia pastoris, or Yarrowia lipolytica are also easily transformed with plasmids which express the viroid RNA scaffold (full-length or deleted) with the inserted RNA of interest and the eggplant tRNA ligase using the appropriate promoters for the yeast species.

In plant tissues, the ELVd RNA and tRNA ligase are expressed using Agrobacterium tumefaciens. Once cloned in binary plasmids that replicate in E. coli and A. tumefaciens under the control of the appropriate promoters (for example Cauliflower mosaic virus 35S promoter), both constructs are delivered into the plant tissue through agroinfiltration. Alternatively, viral vectors are used to express ELVd RNA and tRNA ligase in plant tissues. Suitable viral vectors include vectors derived from Tobacco mosaic virus, Cowpea mosaic virus or Potato virus X.

In insect cells, baculovirus expression vectors (BEV) are used to express ELVd RNA and tRNA ligase.

In mammalian cells, ELVd RNA and tRNA ligase are expressed using any suitable mammalian expression vector. A range of vectors are suitable for this purpose, including vectors derived from mammalian viruses, such as Simian Viruses 40 (SV40), polyomavirus, herpesvirus and papovirus, non-integrating viral vectors (NIVVs), such as adenoviral vectors, adeno-associated viral vectors (AAVVs), and lentiviral vectors (LVs), and plasmids containing the human cytomegalovirus (CMV) promoter. The most widely used host mammalian cell for expression are Chinese hamster ovary (CHO) cells and mouse myeloma cells, including NS0 and Sp2/0 cells.

### 2.6 Production of Two RNAs with Potential Interfering Activity against Human Hepatitis C Virus (HCV)

Two different hybrid molecules were produced consisting of the deleted form of ELVd (ELVdI3D1, 215 nt) acting as a scaffold and two small RNAs (27 and 48-nt-long, here termed HCV1 and HCV2, respectively) inserted between positions U245 and U246 of the ELVd-AJ536613 molecule. The two small RNAs are candidate molecules to interfere with HCV replication. For a review of this kind of antiviral strategies, see (Lee et al., 2013). To test whether large amounts of the chimeric RNAs could be generated, we produced cDNAs corresponding to these two RNAs by PCR. cDNAs were flanked with sites for the type-IIS restriction enzyme Eco31I. Digestion of the cDNAs with this enzyme allowed cloning in the plasmid vector pLELVdI3D1-BZB digested with BpiI (another type-IIS restriction enzyme). The resulting plasmids (pLELVdI3D1-HCV1 and pLELVdI3D1-HCV2) were co-transformed along with p15LtRnlSm in E. coli BL21. Co-transformant colonies were selected in plates with ampicillin and chloramphenicol. 250-ml cultures were inoculated at an optic density at 600 nm (OD₆₀₀) of 0.1 and grown in Terrific Broth (TB) at 37° C. Cells were harvested after 8 h, resuspended in water and frozen.

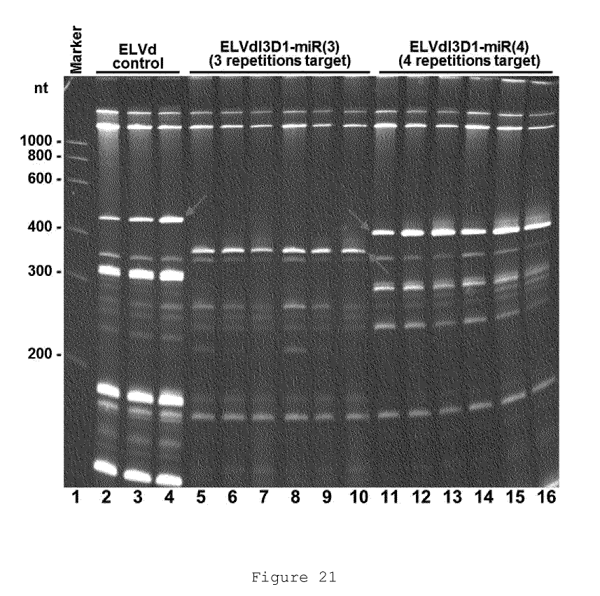

Cells were then broken by treatment with phenol:chloroform and RNAs recovered in the aqueous phase by centrifugation. Aqueous phase was re-extracted with chloroform and loaded into a DEAE Sepharose chromatography column (5 ml column volume). RNA was eluted from the column in the presence of 1 M NaCl. RNAs in the peak fractions were recovered by isopropanol precipitation and further purified by size exclusion chromatography (see below). We finally provided 100 μg of ELVdI3D1-HCV1 and 25 μg of ELVdI3D1-HCV2, as well as a control sample consisting of the deleted form of the viroid with no insert (ELVdI3D1) (FIG. 20).

### 2.7 Production of a MicroRNA Sponge

Large amounts of a chimeric RNA consisting of the deleted form of ELVd (ELVdI3D1) including, between positions U245 a U246 of the viroid molecule, tandem repeats of the target of a human microRNA (miRNA) were produced. This miRNA is overexpressed in certain tumor cells and is a potential therapeutic agent (see Moshiri et al., 2014). First, we constructed cDNAs corresponding to 76 and 100-nt-long RNAs containing three or four tandem repeated targets for the miRNA. The cDNAs were flanked with Eco31I sites, which allowed cloning in pLELVdI3D1-BZB to obtain plasmids pLELVdI3D1-miR(3) and pLELVdI3D1-miR(4). Plasmids were transformed in E. coli BL21 with p15LtRnlSm and colonies selected. Independent E. coli clones were tested to check production of the recombinant RNAs (FIG. 21). We decided to continue with one of the clones producing the RNA of interest with four repetitions. A large E. coli culture was grown and the recombinant RNA purified as described before (phenol:chloroform extraction, DEAE Sepharose chromatography and size exclusion chromatography). We obtained 0.87 mg of the chimeric species ELVdI3D1-miR(4) (315 nt).

### 2.8 Production of Four Hairpin RNAs with Potential Insecticide Activity

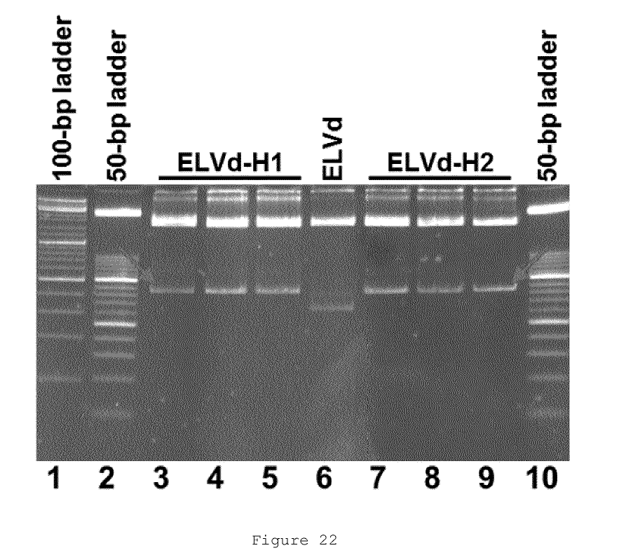

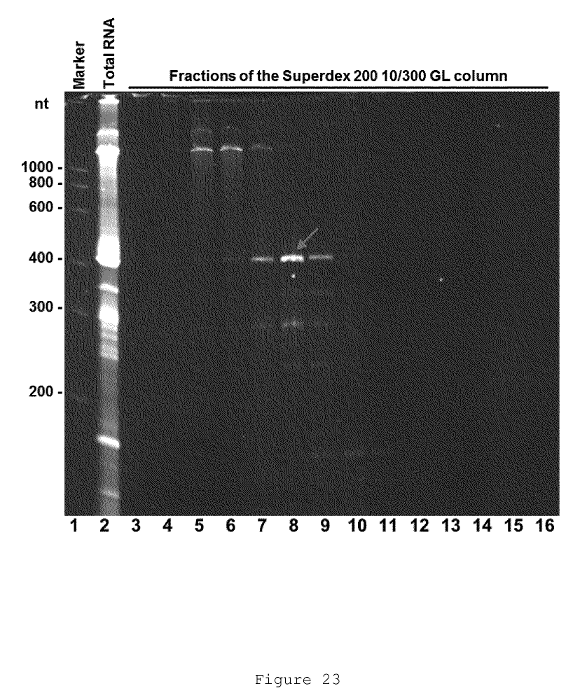

Four hybrid molecules consisting on full-length ELVd with four different hairpins inserted between positions U245 and U246 of ELVd-AJ536613 were produced. Sizes of hairpin RNAs are 100 nt (H1 and H2), 434 nt (H3) and 482 nt (H4). These hairpin RNAs exhibit a very strong secondary structure and may display crop protecting activity (see for example Zhang et al., 2015). We have obtained cDNAs coding for H1 and H2 by PCR and cDNAs coding for H3 and H4 through a commercial gene synthesis provider (GenScript USA Inc). All these cDNAs were flanked by Eco31I recognition sites, which were used to transfer them to vector pLELVd-BZB properly cut with BpiI. The resultant plasmids pLELVd-H1, -H2, -H3 and -H4 were selected by a combination of partial sequencing and analysis of electrophoretic mobility (FIG. 22). Plasmids have been co-transformed in E. coli BL21, along with p15LtRnlSm, and colonies selected in the appropriate antibiotics (ampicillin and chloramphenicol).

Following overproduction of the chimeric RNAs in E. coli, anti-pest activity may be tested.

### 2.9 Purification by Chromatography of the Recombinant RNA Produced in E. Coli

Large amounts of recombinant RNA produced in E. coli using the ELVd-derived system were purified as described above by breaking E. coli cells using phenol:chloroform (1:1) and extracting total RNA in an aqueous buffer. The extract was further purified by re-extraction with chloroform and loaded into a DEAE Sepharose column (column volume 5 ml). RNAs were then eluted from the column with 50 mM Tris-HCl, pH 6.5, 1 M NaCl, 0.2 mM EDTA, collecting 10 fractions of 5 ml. The fractions containing most of the RNA were then subjected to precipitation with isopropanol.

Initially, polyacrylamide gel electrophoresis was used to further purify the circular and linear forms of the recombinant RNAs. However, the high yields (several milligrams) of recombinant RNA produced by the methods described above preclude the use of this technique in the purification scheme. For this reason, size exclusion chromatography was used to separate the RNAs of interest from other E. coli RNAs.

After initial DEAE chromatography purification, RNA was resuspended in a total of 500 μl of buffer 50 mM Tris-HCl pH 6.5, 1 M NaCl, 0.2 mM EDTA, centrifuged to remove non completely dissolved material (10 min at 13,000 rpm), and loaded onto a Superdex 200 10/300 GL column (GE Healthcare) previously equilibrated in the same buffer. RNA was fractionated using the same buffer and collecting 1-ml fractions. Aliquots of the fractions were analyzed by denaturing PAGE in the presence of 8 M urea. FIG. 4 shows that the recombinant RNA, in this case ELVdI1D3-miR(4) (315 nt, see above), was reasonably separated from background RNAs from E. coli. In contrast to PAGE, chromatography allowed purification of the milligram yields of RNA produced in E. coli using the above methods.

### 3. Sequences

| SEQ ID NO: 1 | |
|---|---|
| 1 | gggtggtgtg tgccacccct gatgagaccg aaaggtcgaa atggggtttc gccatgggtc |
| 61 | gggactttaa attcggagga ttcgtccttt aaacgttcct ccaagagtcc cttccccaaa |
| 121 | cccttacttt gtaagtgtgg ttcggcgaat gtaccgtttc gtcctttcgg actcatcagg |
| 181 | gaaagtacac actttccgac ggtgggttcg tcgacacctc tccccctccc aggtactatc |
| 241 | ccctttcaag gatgtgttcc ctaggagggt gggtgtacct cttttggatt gctccggcct |
| 301 | tccaggagag atagaggacg acctctcccc ata |
| ELVd (AJ536613.1 GI: 29825431) | |
| SEQ ID NO: 2 | |
| 1 | tttattagaa caagaagtga ggatatgatt aaactttgtt tgacgaaacc aggtctgttc |
| 61 | cgactttccg actctgagtt tcgacttgtg agagaaggag gagtcgtggt gaacttttat |
| 121 | taaaaaaatt agttcactcg tcttcaatct cttgatcact tcgtctcttc agggaaagat |
| 181 | gggaagaaca ctgatgagtc tcgcaaggtt tactcctcta tcttcattgt ttttttacaa |
| 241 | aatcttg |
| ASBVd (NC_001410.1 GI: 11496574) | |
| SEQ ID NO: 3 | |
| 1 | ggcacctgac gtcggtgtcc tgatgaagat ccatgacagg atcgaaacct cttccagttt |
| 61 | cggcttgtgt gggagtaaag ctttcgctct ctccacagcc tcatcaggaa acccacttca |
| 121 | ggtctcgact ggaaggtcgt taaacttccc ctctaagcgg agtagaggta aatacctccg |
| 181 | tccaaccccg ggaggaaagg ggttgggacc cggaacagat ccagttccgg tcctttggag |
| 241 | tccatttctc tcgttggata ttctcctcgg agaagggaga tggggccagt cccagtcggt |
| 301 | tcgctctcgt agtcacagcc actggggaac ctaggcagat ggctggacgg agtcttagtc |
| 361 | cactccagag gaccttgggt ttgaaacccc caagaggtc |
| CChMVd (NC_003540.1 GI: 20095240) | |
| SEQ ID NO: 4 | |
| 1 | gtcataagtt tcgtcgcatt tcagcgactc atcagtgggc ttagcccaga cttatgagag |
| 61 | agtaaagacc tctcagcccc tccaccttgg ggtgccctat tcggagcact gcagttcccg |
| 121 | atagaaaggc taagcacctc gcaatgaggt aaggtgggac ttttccttct ggaaccaagc |
| 181 | ggttggttcc gaggggggtg tgatccaggt accgccgtag aaactggatt acgacgtcta |
| 241 | cccgggattc aaacccgtcc cctccagaag tgattctgga tgaagagtct gtgctaagca |
| 301 | cactgacgag tctctgagat gagacgaaac tcttctt |
| PLMVd (NC_003636.1 GI: 20177433) | |
| SEQ ID NO: 5 | |
| 1 | msvshrviys fthyklynls sslsslpsri ffpfqspsfh tfsslmpnnq erggyegkkw |
| 61 | qvrpssnrvp gsssnvepvs aataeaitdr lksvditesg aqssvpvtsl qfgsvglapq |
| 121 | spvqhqkviw kpksygtvsg apvveagktp veqksallsk lfkgnllenf tvdnstfsra |
| 181 | qvratfypkf eneksdqeir trmiemvskg laivevtlkh sgslfmyagh eggayaknsf |
| 241 | gniytavgvf vlgrmfreaw gtkaskkqae fneflernrm cismelvtav lgdhgqrprd |
| 301 | dyavvtavte lgngkptfys tpdviafcre wrlptnhvwl fstrksvtsf faaydalcee |
| 361 | gtattvceal sevadisvpg skdhikvqge ileglvariv kressehmer vlrdfpppps |
| 421 | egegldlgpt lreicaanrs ekqqikallq sagtafcpny ldwfgdensg shsrnadrsv |
| 481 | vskflqshpa dlytgkiqem vrlmrekrfp aafkchynlh kindvssnnl pfkmvihvys |
| 541 | dsgfrryqke mrhkpglwpl yrgffvdldl fkvnekktae magsnnqmvk nveednslad |
| 601 | edanlmvkmk fltyklrtfl irnglstlfk egpsayksyy lrqmkiwnts aakqrelskm |
| 661 | ldewavyirr kygnkplsss tylseaepfl eqyakrspqn haligsagnf vkvedfmaiv |
| 721 | egedeegdle pakdiapssp sistrdmvak negliiffpg ipgcaksalc keilnapggl |
| 781 | gddrpvnslm gdlikgrywq kvaderrrkp ysimladkna pneevwkqie nmclstgasa |
| 841 | ipvipdsegt etnpfsidal avfifrvlhr vnhpgnldks spnagyvmlm fyhlydgksr |
| 901 | qefeselier fgslvripvl kpersplpds vrsiieegls lyrlhttkhg rlestkgtyv |
| 961 | qewvkwekql rdillgnady lnsiqvpfef avkevleqlk viargeyavp aekrklgsiv |
| 1021 | faaislpvpe ilgllndlak kdpkvgdfik dksmessiqk ahltlahkrs hgvtavanyg |
| 1081 | sflhqkvpvd vaallfsdkl aaleaepgsv egekinskns wphitlwsga gvaakdantl |
| 1141 | pqllsqgkat ridinppvti tgtleff |

Solanum melongena tRNA ligase AFK76482.1 GI: 388604525

Sequence of plasmid pLELVd (2050 bp)
SEQ ID NO: 6
CGATGCTTCTTTGAGCGAACGATCAAAAATAAGTGCCTTCCCATCAAAAA
AATATTCTCAACATAAAAAACTTTGTGTAATACTTGTAACGCTGCCCCAT
AGGGTGGTGTGTGCCACCCCTGATGAGACCGAAAGGTCGAAATGGGGTTT
CGCCATGGGTCGGGACTTTAAATTCGGAGGATTCGTCCTTTAAACGTTCC
TCCAAGAGTCCCTTCCCCAAACCCTTACTTTGTAAGTGTGGTTCGGCGAA
TGTACCGTTTCGTCCTTTCGGACTCATCAGGGAAAGTACACACTTTCCGA
CGGTGGGTTCGTCGACACCTCTCCCCCTCCCAGGTACTATCCCCTTTCAA
GGATGTGTTCCCTAGGAGGGTGGGTGTACCTCTTTTGGATTGCTCCGGCC
TTCCAGGAGAGATAGAGGACGACCTCTCCCCATAGGGTGGTGTGTGCCAC

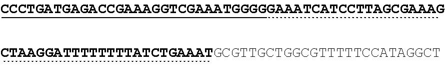

CCGCCCCCCTGACGAGCATCACAAAAATCGACGCTCAAGTCAGAGGTGGC
GAAACCCGACAGGACTATAAAGATACCAGGCGTTTCCCCCTGGAAGCTCC
CTCGTGCGCTCTCCTGTTCCGACCCTGCCGCTTACCGGATACCTGTCCGC
CTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAA
CCCCCCGTTCAGCCCGACCGCTGCGCCTTATCCGGTAACTATCGTCTTGA
GTCCAACCCGGTAAGACACGACTTATCGCCACTGGCAGCAGCCACTGGTA
ACAGGATTAGCAGAGCGAGGTATGTAGGCGGTGCTACAGAGTTCTTGAAG
TGGTGGCCTAACTACGGCTACACTAGAAGGACAGTATTTGGTATCTGCGC
TCTGCTGAAGCCAGTTACCTTCGGAAAAAGAGTTGGTAGCTCTTGATCCG
GCAAACAAACCACCGCTGGTAGCGGTGGTTTTTTTGTTTGCAAGCAGCAG

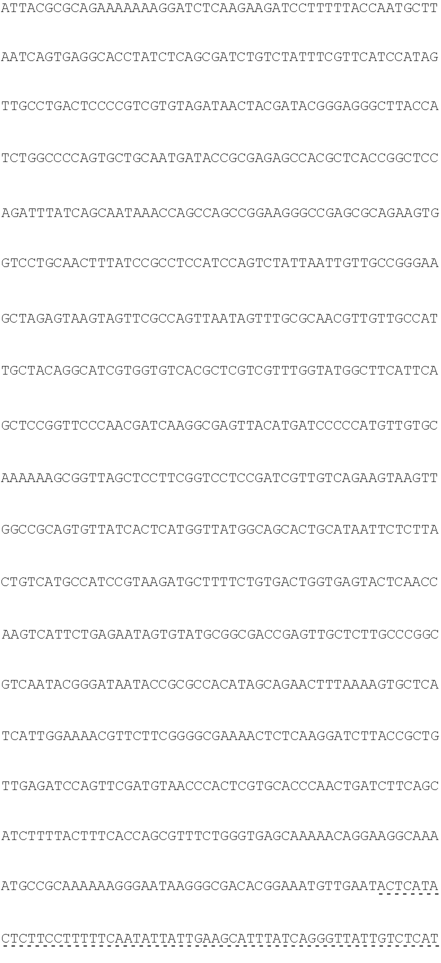

E. coli murein lipoprotein promoter is in italics and ELVd cDNA (C327 to G46 of AJ536613) is in black with the repeated hammerhead ribozyme domain on yellow background. Ribozymes self-cleavage sites are underlined. E. coli ribosomal rrnC terminator is in dotted underline. pUC replication origin is in gray and ampicillin resistance gene (inverse orientation) on gray background (promoter is in dashed underline).

SEQ ID NO: 7
CGATGCTTCTTTGAGCGAACGATCAAAAATAAGTGCCTTCCCATCAAAAAAATATTCTCAACATAAAAAACTTTGTG
TAATACTTGTAACGCTGCCCCATAGGGTGGTGTGTGCCACCCCTGATGAGACCGAAAGGTCGAAATGGGGTTTCGCC
ATGGGTCGGGACTTTAAATTCGGAGGATTCGTCCTTTAAACGTTCCTCCAAGAGTCCCTTCCCCAAACCCTTACTTT
GTAAGTGTGGTTCGGCGAATGTACCGTTTCGTCCTTTCGGACTCATCAGGGAAAGTACACACTTTCCGACGGTGGGT
TCGTCGACACCTCTCCCCCTCCCAGGTACTATCCCCTTGACGCAACTGAATGAAATGGTGAAGGACGGGTCCAGGTG
TGGCTGCTTCGGCAGTGCAGCTTGTTGAGTAGAGTGTGAGCTCCGTAACTAGTCGCGTCTCAAGGATGTGTTCCCTA
GGAGGGTGGGTGTACCTCTTTTGGATTGCTCCGGCCTTCCAGGAGAGATAGAGGACGACCTCTCCCCATAGGGTGGT
GTGTGCCACCCCTGATGAGACCGAAAGGTCGAAATGGGGGAAATCATCCTTAGCGAAAGCTAAGGATTTTTTTTATC
TGAAATGCGTTGCTGGCGTTTTTCCATAGGCTCCGCCCCCCTGACGAGCATCACAAAAATCGACGCTCAAGTCAGAG
GTGGCGAAACCCGACAGGACTATAAAGATACCAGGCGTTTCCCCCTGGAAGCTCCCTCGTGCGCTCTCCTGTTCCGA
CCCTGCCGCTTACCGGATACCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCCGTTCAGCCCGACCGCTGCGC
CTTATCCGGTAACTATCGTCTTGAGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGCCTAACTACGGCTACACTAGAAGG
ACAGTATTTGGTATCTGCGCTCTGCTGAAGCCAGTTACCTTCGGAAAAAGAGTTGGTAGCTCTTGATCCGGCAAACA
AACCACCGCTGGTAGCGGTGGTTTTTTTGTTTGCAAGCAGCAGATTACGCGCAGAAAAAAAGGATCTCAAGAAGATC
CTTTTTACCAATGCTTAATCAGTGAGGCACCTATCTCAGCGATCTGTCTATTTCGTTCATCCATAGTTGCCTGACTC
CCCGTCGTGTAGATAACTACGATACGGGAGGGCTTACCATCTGGCCCCAGTGCTGCAATGATACCGCGAGAGCCACG
CTCACCGGCTCCAGATTTATCAGCAATAAACCAGCCAGCCGGAAGGGCCGAGCGCAGAAGTGGTCCTGCAACTTTAT
CCGCCTCCATCCAGTCTATTAATTGTTGCCGGGAAGCTAGAGTAAGTAGTTCGCCAGTTAATAGTTTGCGCAACGTT
GTTGCCATTGCTACAGGCATCGTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGTCCTCCGATCGTTGTCAGAAGTA
AGTTGGCCGCAGTGTTATCACTCATGGTTATGGCAGCACTGCATAATTCTCTTACTGTCATGCCATCCGTAAGATGC
TTTTCTGTGACTGGTGAGTACTCAACCAAGTCATTCTGAGAATAGTGTATGCGGCGACCGAGTTGCTCTTGCCCGGC
GTCAATACGGGATAATACCGCGCCACATAGCAGAACTTTAAAAGTGCTCATCATTGGAAAACGTTCTTCGGGGCGAA
AACTCTCAAGGATCTTACCGCTGTTGAGATCCAGTTCGATGTAACCCACTCGTGCACCCAACTGATCTTCAGCATCT
TTTACTTTCACCAGCGTTTCTGGGTGAGCAAAAACAGGAAGGCAAAATGCCGCAAAAAAGGGAATAAGGGCGACACG
GAAATGTTGAATACTCATACTCTTCCTTTTTCAATATTATTGAAGCATTTATCAGGGTTATTGTCTCAT
plasmid pLELVd-Spinach (2148 bp). Spinach insert between T245 and T246 of
ELVd highlighted.
SEQ ID NO: 8
CGATGCTTCTTTGAGCGAACGATCAAAAATAAGTGCCTTCCCATCAAAAAAATATTCTCAACATAAAAAACTTTGTG
TAATACTTGTAACGCTGCCCCATAGGGTGGTGTGTGCCACCCCTGATGAGACCGAAAGGTCGAAATGGGGTTTCGCC
ATGGGTCGGGACTTTAAATTCGGAGGATTCGTCCTTTAAACGTTCCTCCAAGAGTCCCTTCCCCAAACCCTTACTTT
GTAAGTGTGGTTCGGCGAATGTACCGTTTCGTCCTTTCGGACTCATCAGGGAAAGTACACACTTTCCGACGGTGGGT
TCGTCGACACCTCTCCCCCTCCCAGGTACTATCCCCTTCCGACCAGAATCATGCAAGTGCGTAAGATAGTCGCGGGC
CGGTCAAGGATGTGTTCCCTAGGAGGGTGGGTGTACCTCTTTTGGATTGCTCCGGCCTTCCAGGAGAGATAGAGGAC
GACCTCTCCCCATAGGGTGGTGTGTGCCACCCCTGATGAGACCGAAAGGTCGAAATGGGGGAAATCATCCTTAGCGA
AAGCTAAGGATTTTTTTTATCTGAAATGCGTTGCTGGCGTTTTTCCATAGGCTCCGCCCCCCTGACGAGCATCACAA
AAATCGACGCTCAAGTCAGAGGTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCCTTCGGGAAGCGTGGCGCTT
TCTCATAGCTCACGCTGTAGGTATCTCAGTTCGGTGTAGGTCGTTCGCTCCAAGCTGGGCTGTGTGCACGAACCCCC
CGTTCAGCCCGACCGCTGCGCCTTATCCGGTAACTATCGTCTTGAGTCCAACCCGGTAAGACACGACTTATCGCCAC
TGGCAGCAGCCACTGGTAACAGGATTAGCAGAGCGAGGTATGTAGGCGGTGCTACAGAGTTCTTGAAGTGGTGGCCT
AACTACGGCTACACTAGAAGGACAGTATTTGGTATCTGCGCTCTGCTGAAGCCAGTTACCTTCGGAAAAAGAGTTGG
TAGCTCTTGATCCGGCAAACAAACCACCGCTGGTAGCGGTGGTTTTTTTGTTTGCAAGCAGCAGATTACGCGCAGAA
AAAAAGGATCTCAAGAAGATCCTTTTTACCAATGCTTAATCAGTGAGGCACCTATCTCAGCGATCTGTCTATTTCGT
TCATCCATAGTTGCCTGACTCCCCGTCGTGTAGATAACTACGATACGGGAGGGCTTACCATCTGGCCCCAGTGCTGC
AATGATACCGCGAGAGCCACGCTCACCGGCTCCAGATTTATCAGCAATAAACCAGCCAGCCGGAAGGGCCGAGCGCA
GAAGTGGTCCTGCAACTTTATCCGCCTCCATCCAGTCTATTAATTGTTGCCGGGAAGCTAGAGTAAGTAGTTCGCCA
GTTAATAGTTTGCGCAACGTTGTTGCCATTGCTACAGGCATCGTGGTGTCACGCTCGTCGTTTGGTATGGCTTCATT
CAGCTCCGGTTCCCAACGATCAAGGCGAGTTACATGATCCCCCATGTTGTGCAAAAAAGCGGTTAGCTCCTTCGGTC
CTCCGATCGTTGTCAGAAGTAAGTTGGCCGCAGTGTTATCACTCATGGTTATGGCAGCACTGCATAATTCTCTTACT
GTCATGCCATCCGTAAGATGCTTTTCTGTGACTGGTGAGTACTCAACCAAGTCATTCTGAGAATAGTGTATGCGGCG
ACCGAGTTGCTCTTGCCCGGCGTCAATACGGGATAATACCGCGCCACATAGCAGAACTTTAAAAGTGCTCATCATTG
GAAAACGTTCTTCGGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGCAAAATGCCGCAAA
AAAGGGAATAAGGGCGACACGGAAATGTTGAATACTCATACTCTTCCTTTTTCAATATTATTGAAGCATTTATCAGG
GTTATTGTCTCAT
plasmid pLELVd-Strep (2092 bp). Strep insert between T245 and T246 of ELVd
highlighted.

### 4. REFERENCES

Abelson J. et al (1998). J. Biol. Chem. 273: 12685-12688.

Batey R. T. (2014). Curr. Opin. Struct. Biol. 26C: 1-8.

Breaker R. R. (2004) Nature 432: 838-845.

Citartan M. et al (2012). Biosens. Bioelectron. 34: 1-11.

Daròs J. A., Elena S. F. and Flores R. (2006) EMBO Rep. 7: 593-598.

Ding B. (2010). Wiley Interdiscip. Rev. RNA 1: 362-375.

Elena S. F. et al (2009) Viruses 1: 241-254.

Ellington A. D. et al (1990). Nature 346: 818-822.

Engler C. et al (2014). Methods Mol. Biol. 1116: 119-131.

Englert M. et al (2007). Biochimie 89: 1351-1365.

Fadda Z. et al (2003). J. Virol. 77: 6528-6532.

Flores R. et al (2005). Annu. Rev. Phytopathol. 43: 117-139.

Germer K. et al (2013) Int. J. Biochem. Mol. Biol. 4: 27-40.

Gold L., et al (2012). Cold Spring Harb. Perspect. Biol. 4:

[symbol]

Gömez G. and Pallás V. (2010). PLoS One 5: e12269.

Holt C. E. and Schuman E. M. (2013). Neuron 80: 648-657.

Isaacs F. J., et al (2006). Nat. Biotechnol. 24: 545-554.

Lee C. H. et al (2013) World J. Gastroenterol. 19: 8949-8962.

Martínez F. et al (2009). J. Gen. Virol. 90: 3057-3065.

Milligan J. F. et al (1987). Nucleic Acids Res. 15: 8783-8798.

Moshiri F. et al (2014). Gastroenterol. Hepatol. Bed Bench. 7: 43-54.

Nakamura Y. et al (2012). Genes Cells 17: 344-364.

Navarro B. et al (2012). Biochimie 94: 1474-1480.

Nohales M. A. et al (2012a). PNAS USA 109: 13805-13810.

Nohales M. A. et al (2012b). J. Virol. 86: 8269-8276.

Paige J. S. et al (2011). Science 333: 642-646.

Ponchon L. et al (2009). Nat. Protoc. 4: 947-959.

Ponchon L. et al (2013). Nucleic Acids Res. 41: e150.

Ponchon L. and Dardel F. (2007). Nat. Methods 4: 571-576.

Ponchon L. and Dardel F. (2011). Methods 54: 267-273.

Roberts T. C. and Wood M. J. (2013). Essays Biochem. 54: 127-145.

Robertson D. L. and Joyce G. F. (1990). Nature 344: 467-468.

Rodrigo G. et al (2013). PLoS Comput. Biol. 9: e1003172.

Sabin L. R., Delás M. J. and Hannon G. J. (2013). Mol. Cell. 49: 783-794.

Schumacher J. et al (1983). Anal. Biochem. 135: 288-295.

Shaner N. C. et al (2004). Nat. Biotechnol. 22: 1567-1572.

Srisawat C. and Engelke D. R. (2001). RNA 7: 632-641.

Srisawat C. and Engelke D. R. (2002). Methods 26: 156-161.

Sundaram P. et al (2013). Eur. J. Pharm. Sci. 48: 259-271.

Tuerk C. and Gold L. (1990). Science 249: 505-510.

Umekage S. and Kikuchi Y. (2009a). J. Biotechnol. 139: 265-272.

Umekage S. and Kikuchi Y. (2009b). J. Biosci. Bioeng. 108: 354-356.

Zhang J. et al (2015). Science 347: 991-994.

Zhou J. et al (2012). Front. Genet. 3: 234.

1. A method of RNA production comprising;
expressing in a host cell;
a nucleic acid encoding a chimeric RNA molecule comprising a target RNA and a plant viroid scaffold; and,
a nucleic acid encoding a tRNA ligase.

2. A method according to claim 1 comprising allowing said chimeric RNA molecule to accumulate in the host cell.

3. A method according to any one of the preceding claims comprising isolating and/or purifying the chimeric RNA molecule from the host cell.

4. A method according to claim 3 comprising separating the target RNA from the recombinant RNA molecule.

5. A method according to any one of the preceding claims wherein the host cell is a prokaryotic cell.

6. A method according to claim 5 wherein the host cell is an E. coli cell

7. A method according to any one of the preceding claims wherein the nucleic acids are heterologous to the host cell.

8. A method according to any one of the preceding claims wherein the target RNA is inserted within the plant viroid scaffold in the chimeric RNA molecule.

9. A method according to claim 8 wherein the target RNA is inserted within the plant viroid scaffold outside the hammerhead ribozyme domain.

10. A method according to claim 9 wherein the target RNA is inserted into the viroid scaffold at a position corresponding to position 245-246 of ELVd.

11. A method according to any one of the preceding claims wherein the chimeric RNA molecule produced by the host cells is monomeric.

12. A method according to any one of the preceding claims wherein the target RNA is 5 to 1000 ribonucleotide bases in length.

13. A method according to any one of the preceding claims wherein the target RNA is an RNA aptamer.

14. A method according to any one of the preceding claims wherein the plant viroid scaffold comprises all or part of a plant viroid.

15. A method according to any one of the preceding claims wherein the plant viroid is an Avsunviroidae viroid.

16. A method according to any one of the preceding claims wherein the plant viroid is Avocado sunblotch viroid (ASBVd), Peach latent mosaic viroid (PLMVd), Chrysanthemum chlorotic mottle viroid (CChMVd) or Eggplant latent viroid (ELVd

[symbol]

17. A method according to claim 16 wherein the plant viroid is Eggplant latent viroid (ELVd).

18. A method according to any one of claims 14 to 16 wherein the plant viroid scaffold comprises or consists of part of the full-length plant viroid.

19. A method according to claim 18 wherein the plant viroid scaffold comprises a full-length plant viroid with the regions corresponding to bases 56 to 116 and 214 to 310 of ELVd deleted.

20. A method according to claim 18 wherein the plant viroid scaffold comprises a full-length plant viroid with the regions corresponding to bases 56 to 141 and 279 to 310 of ELVd deleted.

21. A method according to any one of the preceding claims wherein the plant viroid scaffold comprises a nucleotide sequence having at least 60% identity to one or more of; the sequence of bases 1 to 55, 142 to 278 and 311 to 311 of SEQ ID NO: 1; the sequence of bases 1 to 55, 117 to 213, and 311 to 333 of SEQ ID NO: 1; and the sequences of any one of SEQ ID NOS: 1 to 4.

22. A method according to any one of the preceding claims wherein the tRNA ligase is a plant tRNA ligase.

23. A method according to claim 22 wherein the tRNA ligase is a plant chloroplast tRNA ligase.

24. A method according to claim 22 or claim 23 wherein the tRNA ligase is eggplant tRNA ligase or an orthologue thereof.

25. A method according to any one of the preceding claims wherein the tRNA ligase comprises an amino acid sequence having at least 60% sequence identity to SEQ ID NO: 5.

26. A method according to any one of the preceding claims wherein the tRNA ligase is constitutively expressed in the cell

27. A method according to any one of the preceding claims wherein the nucleic acids are contained in expression vectors.

28. A method according to any one of the preceding claims wherein the method comprises introducing the nucleic acids or expression vectors into the host cell.

29. An isolated nucleic acid encoding a chimeric RNA molecule chimeric RNA molecule comprising a target RNA and a plant viroid scaffold.

30. A vector comprising the isolated nucleic acid of claim 29 and optionally a nucleic acid encoding a tRNA ligase.

31. A vector comprising a nucleic acid sequence encoding a plant viroid scaffold, said nucleic acid sequence comprising a cloning site for insertion of a heterologous nucleotide sequence encoding a target RNA into the nucleic acid sequence.

32. A set of vectors comprising a first vector comprising an isolated nucleic acid encoding a chimeric RNA molecule and a second vector comprising a nucleic acid encoding a tRNA ligase.

33. A host cell that expresses;
a chimeric RNA molecule comprising a target RNA and a plant viroid scaffold, and;
a tRNA ligase.

34. A host cell according to claim 33 comprising a nucleic acid encoding the tRNA ligase and a nucleic acid encoding the chimeric RNA molecule.

35. A host cell comprising an isolated nucleic acid, vector or set of vectors according to any one of claims 29 to 32.

36. A system for the production of RNA comprising;
a host cell,
a nucleic acid encoding a chimeric RNA molecule comprising a target RNA and a plant viroid scaffold, and
a nucleic acid encoding a tRNA ligase.

37. A system according to claim 36 for use in a method according to any one of claims 1 to 28.

38. A kit for the production of RNA comprising;
a nucleic acid encoding a chimeric RNA molecule comprising a target RNA and a plant viroid scaffold; or
a nucleic acid encoding a plant viroid scaffold,
said nucleic acid comprising a cloning site for insertion of a target RNA into the plant viroid scaffold.

39. A kit according to claim 40 further comprising a nucleic acid encoding a tRNA ligase.

40. A kit according to claim 38 further comprising a host cell that expresses a heterologous nucleic acid encoding a tRNA ligase.

41. A kit comprising an isolated nucleic acid, vector, set of vectors, or host cell according to any one of claims 29 to 35
